SEQUENCE LISTING (7 sequences)

SEQ ID NO: 1 - protein, 282 aa, Lucilia sericata
MKTFIALSLFVAIASAGILPAVEQRLPLVPVMPLEELEGRITNGELAKPGQFPYQAGLSLVFGNKGAWCGGTLISNRWVLTAAHCTDGADGVTVYLGATDIKNDNEKGQQRIYSSKANIVVHANWDASTLSNDISLIKLPVAVEFNELIQPATLPKMDGKYSTYEGDMVWASGWGRDSDAATSVSQFLRYIEVPVLKQSTCKTYYLGSVTDKMICIKSVDKKSTCNGDSGGPLVYKDGGVNYVIGATSFGIALGCEKGWPGVFTRVTSYLDWIEEVSGVVNK

SEQ ID NO: 2 - protein, 242 aa, Lucilia sericata
ITNGELAKPGQFPYQAGLSLVFGNKGAWCGGTLISNRWVLTAAHCTDGADGVTVYLGATDIKNDNEKGQQRIYSSKANIVVHANWDASTLSNDISLIKLPVAVEFNELIQPATLPKMDGKYSTYEGDMVWASGWGRDSDAATSVSQFLRYIEVPVLKQSTCKTYYLGSVTDKMICIKSVDKKSTCNGDSGGPLVYKDGGVNYVIGATSFGIALGCEKGWPGVFTRVTSYLDWIEEVSGVVNK

SEQ ID NO: 3 - protein, 6 aa, Artificial Sequence
  Synthetic peptide
SFLLRN

SEQ ID NO: 4 - protein, 6 aa, Artificial Sequence
  Synthetic peptide
SLIGKV

SEQ ID NO: 5 - protein, 6 aa, Artificial Sequence
  Synthetic peptide
TFRGAP

SEQ ID NO: 6 - protein, 6 aa, Artificial Sequence
  Synthetic peptide
GYPGQV

SEQ ID NO: 7 - protein, 4 aa, Artificial Sequence
  Synthetic peptide
AAPF

(19) United States
(12) Patent Application Publication
(10) Pub. No.: US 2011/0280858 A1
(43) Pub. Date: Nov. 17, 2011

(54) TREATMENT OF WOUNDS

(75) Inventors: David I. Pritchard, Nottingham (GB); Kevin M. Shakesheff, Nottingham (GB); Adele J. Horobin, (US); Alan Brown, Nottingham (GB)

(73) Assignee: The Secretary of State for Defence, Salisbury (GB)

(21) Appl. No.: 13/083,495

(22) Filed: Apr. 8, 2011

Related U.S. Application Data
(63) Continuation-in-part of application No. 11/515,156, filed on Aug. 31, 2006.
Application No. 11/515,156 is a division of application No. 10/111,252, filed on Aug. 23, 2002, now Pat. No. 7,144,721.
Application No. 10/111,252 is a continuation-in-part of application No. 11/823,209, filed on Jun. 27, 2007, now abandoned.
(60) Provisional application No. 60/819,031, filed on Jul. 6, 2006.

(30) Foreign Application Priority Data
Oct. 22, 1999 (GB) 9925005.2

(51) Int. Cl. A61K 38/48; A61P 17/02; C12N 9/76; C07H 21/00; C12N 15/63

(52) U.S. Cl. 424/94.64; 536/23.2; 435/320.1; 435/213

(57) ABSTRACT

The invention generally provides compositions, kits, pharmaceuticals, and methods that promote and enhance wound healing. Such compositions comprise isolated trypsinogen or trypsin polypeptides or isolated polypeptides obtainable from the excretions/secretions (ES) of Lucilia sericata.

### CROSS-REFERENCE TO RELATED APPLICATION

This application is a continuation-in-part of U.S. patent application Ser. Nos. 11/515,156, filed on Aug. 31, 2006, and 11/823,209, filed on Jun. 27, 2007. U.S. patent application Ser. No. 11/515,156 is a division of U.S. patent application Ser. No. 10/111,252, issued as U.S. Pat. No. 7,144,721, which is the U.S. national phase, pursuant to 35 U.S.C. §371, of international application No. PCT/GB00/04034, filed on Oct. 20, 2000, designating the United States and published in English on May 3, 2001 as publication WO 01/31033 A2, which claims priority to application Ser. No. GB 9925005.2, filed on Oct. 22, 1999. U.S. patent application Ser. No. 11/823,209 claims the benefit of U.S. provisional application No. 60/819,031, filed on Jul. 6, 2006. The entire contents of the aforementioned patent applications are incorporated herein by this reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND TO THE INVENTION

Efficient wound healing is a complex physiological process which involves many mechanisms including cell migration, growth factor secretion, angiogenesis, tissue remodelling and the intrinsic proteinase/antiproteinase balance of the wound contributing in concert and in an apparently staged manner to accelerate controlled tissue regeneration.

Wound care products are essential in modern medical practice, especially for the treatment of patients with chronic wounds or burns. Many different substances have previously been proposed as having activities which contribute to the healing of wounds. These previously proposed substances include streptokinase, collagenase and streptodornase (all obtained from bacterial sources), bromelain (from pineapples), plasmin and trypsin (obtained from cattle) and krill enzymes (obtained from Crustacea). Clinical trial data indicate that such substances are only partially effective in promoting the healing of wounds.

The larvae (maggots) of the green bottle fly, Lucilia sericata, are known to have significant wound healing attributes as live organisms. Debridement treatment using the larvae of Lucilia sericata, has become a widely accepted clinical practice. However, little has been reported in the literature about the way in which these larvae go about their task of cleaning wounds to an extent that conventionally untreatable wounds heal.

Although efficacious, live larvae are unpleasant to many patients and the use of live larvae on wounds and the introduction of their crude secretions into wounds, which inevitably occurs when the larvae are used, are unacceptable to many patients and to many medical practitioners. The use of live organisms also increases the risk of infection or allergic reactions in the patient.

It is an object of the invention to overcome at least some of the above problems.

### TECHNICAL FIELD

The present invention relates to the treatment of wounds. More particularly, it relates to substances which promote the healing of wounds, to compositions and to dressings which incorporate such substances and to a method of treating wounds using such substances.

### SUMMARY OF THE INVENTION

An enzyme which occurs naturally in the crude secretions of the larvae of Lucilia sericata was purified and sequenced, which exhibits trypsin-like serine proteinase activity and wound-healing promotion activity. The use of the purified enzyme in the treatment of a wound avoids the need to bring live organisms and/or their crude secretions into contact with the wound.

The present invention provides an isolated protein characterised in that:

i) it is secreted by the organism Lucilia sericata;
ii) it exhibits optimum proteolytic activity against FITC-casein at a pH of 8.0 to 8.5;
iii) it exhibits proteolytic activity against Tosyl-Gly-Pro-Arg-AMC but not against Suc-Ala-Ala-Phe-AMC;
iv) its proteolytic activity against FITC-casein and Tosyl-Gly-Pro-Arg-AMC is inhibited by the serine proteinase inhibitors PMSF and APMSF; and
v) it is bound by immobilised aminobenzamidine.

The presence of at least two such proteins in the excretions/secretions (ES) of Lucilia sericata, one of molecular weight of about 25 kDa and one at about 75 kDa, have been identified.

In a particular embodiment, the protein is a trypsin derivable from a trypsinogen of SEQ ID NO:1.

(SEQ ID NO: 1)
MKTFIALSLFVAIASAGILPAVEQRLPLVPVMPLEELEGRITNGELAKPG
QFPYQAGLSLVFGNKGAWCGGTLISNRWVL [symbol] CTDGADGVTVYLGAT
DIKNDNEKGQQRIYSSKANIVVHANWDASTLSN [symbol] IKLPVAVEFNEL
IQPATLPKMDGKYSTYEGDMVWASGWGRDSDAATSVSQFLRYIEVPVLKQ
STCKTYYLGSVTDKMICIKSVDKKST [symbol] GPLVYKDGGVNYVIGAT
SFGIALGCEKGWPGVFTRVTSYLDWIEEVSGVVNK.

In SEQ ID NO:1, the activation site of the enzyme is indicated in bold and underlined and active site residues are indicated in bold italics and underlined. The trypsinogen set forth in SEQ ID NO:1 has 96% sequence identity with a trypsinogen from Lucilia cuprina, which is surprising since this species is not used in wound healing in any sense and in fact is known to destroy tissue by attacking live tissue in the form of “blow-fly” attacks on livestock. Hence it would not be expected that digestive enzymes such as trypsin from the two species would be so closely related.

Trypsinogens of SEQ ID NO:1 form a further aspect of the invention.

Thus the trypsin derived from the trypsinogen will comprise SEQ ID NO:6

(SEQ ID NO: 6)
ITNGELAKPGQFPYQAGLSLVFGNKGAWCGGTLISNRWVL [symbol] CTDGA
DGVTVYLGATDIKNDNEKGQQRIYSSKANIVVHANWDASTLSN [symbol] IK
LPVAVEFNELIQPATLPKMDGKYSTYEGDMVWASGWGRDSDAATSVSQFL
RYIEVPVLKQSTCKTYYLGSVTDKMICIKSVDKKST [symbol] GPLVYKD
GGVNYVIGATSFGIALGCEKGWPGVFTRVTSYLDWIEEVSGVVNK.

The invention also provides an isolated protein, characterised in that it is a functional homologue of the protein of the invention. Such proteins will have a trypsinogen or trypsin activity and generally have homologous amino acid sequences to trypsins derivable from the trypsinogen of SEQ ID NO:1 or the trypsin of SEQ ID NO:6 in as much as they have at least 96% sequence identity, preferably at least 98% sequence identity and more preferably at least 99% sequence identity to SEQ ID NO:1 or SEQ ID NO:6. Alternatively, they may comprise fragments of the protein that have one or more of the desired activities as listed hereinafter. Suitably fragments will comprise at least 10, for instance at least 20 and in particular at least 30 contiguous amino acids from SEQ ID NO:1. In particular, any fragment will comprise all the active sites (i.e. those regions shown in bold italics in SEQ ID NO: 5) of the base sequence, arranged in a similar configuration to how they appear in that Sequence.

Typically the protein will be a naturally produced protein secreted by an insect in its larval stage. Suitable insects are envisaged as being those that are used in their larval form in the treatment of wounds.

It is also envisaged that clones of either the Lucilia protein or functional homologues thereof, will form part of the invention and may be used in wound treatment. In a particular embodiment, the protein is a recombinant protein and in particular a protein of SEQ ID NO:1 or a functional homologue thereof as defined above, which may be activated to form a trypsin in particular of SEQ ID NO:6.

In this regard, the protein of the invention, in particular a trypsinogen, may be expressed in bulk in an active recombinant form, for example in an insect/baculovirus expression system. Thereafter, recovered trypsinogen is suitably activated for example by incubation with a suitable activating enzyme such as trypsin-agarose. Incubation conditions will generally be understood in the art, but will include incubation at 37° C. with an activating concentration of the enzyme for a period of from 30 minutes to 4 hours, for instance for about 1 hour. The activating enzyme is then suitably removed for example by centrifugation, before the trypsin is employed in the compositions and methods of treatment aspects of the invention.

In one aspect, the invention generally provides an isolated polypeptide, analog, or functional fragment thereof, where the polypeptide contains an amino acid sequence that naturally occurs in Lucilia sericata; that exhibits serine protease activity by degrading Tosyl-Gly-Pro-Arf-AMC.HCl; and that has serine protease activity as assayed by Tosyl-Gly-Pro-Arf-AMC.HCl degradation, where the protease activity is reduced by at least about 5%, 10%, 25% or more following incubation with soybean trypsin inhibitor (STI) and/or iminodiacetic acid (IA); and that increases the migration of a cell in a migration assay. In various embodiments, migration is increased by at least about 5%, 10%, 25%, 50%, 75%, or 100%.

In one embodiment, the polypeptide consists essentially of or is a polypeptide isolated from L. sericata. The polypeptide in particular is a trypsin obtainable from the trypsinogen of SEQ ID NO:1 as described above, or a functional homologue or a fragment thereof.

In another embodiment, the polypeptide enhances fibroblast migration in a two-dimensional wound assay. In yet another embodiment, the polypeptide degrades fibronectin into fragments ranging between 29 to 189 kDa.

In still other embodiments, the soybean trypsin inhibitor or iminodiacetic acid reduces the serine protease activity by at least about 5%, 10%, 20%, 25%, 50%, 75% or more relative to a control. In one embodiment, the serine protease activity is assayed at pH 7.3. In another embodiment, cell migration is increased by at least 5%, 10%, 20%, 25%, or 50% at 24 hours relative to a control condition, such as an untreated control culture or wound.

In another aspect, the invention generally features an isolated polypeptide, analog, or functional fragment thereof, where the polypeptide contains an amino acid sequence that naturally occurs in Lucilia sericata; that exhibits metalloprotease activity by degrading an H-Leu-AMC substrate; that has metalloprotease activity as assayed by -Leu-AMC substrate, where the protease activity is reduced by at least 5% following incubation with 1,10 phenanthroline; and that increases cell migration in a migration assay. In one embodiment, the polypeptide consists essentially of a polypeptide isolated from L. sericata. In another embodiment, the polypeptide enhances fibroblast migration by at least 5% in a two-dimensional wound assay.

In another aspect, the invention generally features an isolated peptide fragment of fibronectin or analog thereof, where the peptide is isolated following incubation of fibronectin with at least one polypeptide derived from an L. sericata excretion or secretion or a trypsin derivable from a trypsinogen of SEQ ID NO:1 such as a trypsin of SEQ ID NO:6; between 29 and 189 kDa; and increases the migration of a cell in a migration assay. In one embodiment, the polypeptide increases fibroblast migration by at least 5%, 10%, 25%, 50%, 75%, or 100% in a two-dimensional wound assay. In another embodiment, the peptide has a size that is any one or more of 182, 134, 82, 69, 58, and 29 kDa. In another embodiment, the fibronectin degradation is carried out at pH 7.3. In yet another embodiment, production of the peptide fragment is reduced when the incubation this aspect is carried out in the presence of soybean trypsin inhibitor or iminodiacetic acid. In yet another embodiment, cell migration is increased by at least 10% at 24 hours relative to a control condition.

In a further aspect, the invention relates to a method for treating a wound to promote healing thereof in a human or non-human mammal which comprises applying to the wound a therapeutically effective amount of a sterile composition comprising a protein or polypeptide according to the invention as active ingredient. The invention also relates to the use of a protein of the invention or fragment thereof in the preparation or manufacture of a medicament for the treatment of wounds.

In a further aspect, the invention relates to an isolated peptide selected from the group consisting of; Ser-Phe-Leu-Leu-Arg-Asn (SEQ ID NO: 3); Ser-Leu-Ile-Gly-Lys-Val (SEQ ID NO: 4); Thr-Phe-Arg-Gly-Ala-Pro (SEQ ID NO: 5); Gly-Tyr-Pro-Gly-Gln-Val (SEQ ID NO: 6), and a peptide having an N-terminal sequence selected from: Ser-Phe-Leu-Leu-Arg-Asn (SEQ ID NO: 3); Ser-Leu-Ile-Gly-Lys-Val (SEQ ID NO: 4); Thr-Phe-Arg-Gly-Ala-Pro (SEQ ID NO: 5); or Gly-Tyr-Pro-Gly-Gln-Val (SEQ ID NO: 6), or a protected analogue thereof which is protected against aminopeptidase activity, and to the use of one or more of such peptides in a method for treating a wound to promote healing thereof in a human or non-human mammal which comprises applying to the wound one or more of said peptides. Furthermore, the invention relates to the use of such a peptide in the preparation or manufacture of a medicament for the treatment of wounds.

In one embodiment of the invention, there is provided a dressing for a wound, which comprises a sterile support carrying a therapeutically effective amount of a protein and/or at least one peptide according to the invention.

In yet another aspect, the invention provides a composition for promoting wound healing, the composition containing an effective amount of a polypeptide or peptide fragment of any previous aspect, in a pharmaceutically acceptable excipient. In one embodiment, the composition contains a sterile support that provides a dressing to protect the wound.

In yet another aspect, the invention provides a method for promoting wound healing in a subject in need thereof, the method involving contacting the wound with an effective amount of a polypeptide, analog, or fragment thereof of any previous aspect, where the polypeptide increases fibroblast migration, thereby promoting wound healing.

In yet another aspect, the invention provides a method for increasing cell migration in a subject, the method involving contacting a cell or cell substrate with an effective amount of a polypeptide of a previous aspect, where the polypeptide increases cell migration, thereby promoting wound healing.

In yet another aspect, the invention provides a method for increasing cell motility, the method involving contacting a cell or cell substrate with an effective amount of a polypeptide of a previous aspect, thereby increasing cell motility.

In yet another aspect, the invention provides a method for increasing tissue formation, the method involving contacting a cell or cell substrate with an effective amount of a polypeptide of a previous aspect, thereby increasing tissue formation.

In yet another aspect, the invention provides a method for degrading fibronectin, the method involving contacting a cell or cell substrate with an effective amount of a polypeptide of a previous aspect, thereby degrading fibronectin.

In various embodiments of the previous aspects, the method further involves the step of obtaining the polypeptide.

In another aspect, the invention provides a packaged pharmaceutical containing a polypeptide of any previous aspect; and instructions for using the polypeptide to enhance wound healing in a subject.

In yet another aspect, the invention provides a kit for promoting wound healing in a subject, the kit containing a polypeptide of any previous aspect, and instructions for use thereof.

In yet another aspect, the invention provides a pharmaceutical composition containing an effective amount of an isolated excretory/secretory (ES) composition containing one or more polypeptides of a previous aspect, wherein the effective amount of ES is between 0.01 and 100 μg/ml (e.g., is 0.01, 0.1, 0.25, 0.5, 1, 5, 10, 25, 50, 75, or 100 μg/ml).

In yet another aspect, the invention provides a method for promoting wound healing in a subject in need thereof, the method involving contacting the wound with an effective amount of the pharmaceutical composition of the previous aspect, thereby promoting wound healing.

In various embodiments of any of the above aspects, the polypeptide or peptide fragment is recombinantly expressed or chemically synthesized. In still other embodiments, the polypeptide or peptide fragment is protected (e.g., by the presence of a modification) against aminopeptidase activity. In yet other embodiments of any previous aspect, the cell is a fibroblast or a keratinocyte. In still other embodiments, the method or composition promotes the healing of a chronic wound (e.g., an ulcer) by increasing, for example, cell (e.g., keratinocyte or fibroblast) migration or motility, tissue formation, or by modifying a molecular component of a wound.

### DEFINITIONS

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.

By “analog” is meant a structurally related polypeptide or nucleic acid molecule having the function of a reference polypeptide or nucleic acid molecule.

By “cell migration” is meant the movement of a cell in, over, or through a substrate. Cell migration is typically measured in a cell migration or wound healing assay as described herein.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “degradation” is meant the proteolytic breakdown of a protein into peptide fragments.

By “enhances fibroblast migration” is meant increases a cellular function related to motility.

By “ES” (excretions/secretions or excretory/secretory) is meant a larval excretion or secretion having proteolytic activity. Accordingly, larval secretions or excretions, containing serine proteinase, aspartyl proteinase, and metalloproteinase activities regardless of method of release are expressly included in the term “ES.” Such compositions degrade common extracellular matrix (ECM) components that are present within a wound.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “identity” is meant the amino acid or nucleic acid sequence identity between a sequence of interest and a reference sequence. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “promoting wound healing” is meant increase a biological activity associated with cell motility, migration, or tissue formation. Accordingly, methods that promote wound healing result in a positive alteration in the wound (e.g., a stabilize the wound, decrease wound size or wound pathology). Promoting would healing in accordance with the invention is facilitated by one or more of cell migration, increasing cell motility, increasing tissue formation and degrading fibronectin.

By “increases or decreases” is meant a positive or negative alteration.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In one embodiment, the preparation is at least 75%, 85%, 90%, 95%, or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “naturally occurs” is meant is endogenously expressed in a cell of an organism.

By “obtaining” as in “obtaining the polypeptide” is meant synthesizing, purchasing, or otherwise acquiring the polypeptide.

A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition.

By “wound” is meant any damage to the skin, epidermis or connective tissue whether by injury or by disease and as such is taken to include, but not to be limited to, cuts, punctures, surgical incisions, ulcers, pressure sores, burns, including burns caused by heat, freezing, chemicals, electricity and radiation, dermal abrasion or assault, osteomyelitis and orthopaedic wounds. The wound may be infected. Additionally, the wound may be chronic or acute.

### BRIEF DESCRIPTION OF THE DRAWINGS

FIG. 1 is an elution profile of Lucilia sericata excretory/secretory (ES) having been passed through an affinity column.

FIG. 2 shows the residual activity of the column fractions identified in FIG. 1.

FIG. 3 shows a picture of a gel after electrophoresis of the column fractions identified in FIG. 1.

FIG. 4 shows FITC-Casein hydrolysis by Lucilia sericata ES in the presence of APMSF/PMSF at pH8.

FIGS. 5A and 5B demonstrate Tosyl-Gly-Pro-Arg-AMC (FIG. 5A) and Suc-Ala-Ala-Phe-AMC (FIG. 5B) hydrolysis by Lucilia sericata in the presence of APMSF and PMSF.

FIG. 6 is a series of five photomicrographs showing a quantitative analysis of cell migration. A region within each image, outlined by a black rectangle, was analyzed for HDNF cell surface area coverage. The perimeter of each cell or group of confluent cells lying within the selected region was outlined and the enclosed area (shown in black for illustrative purposes) measured in mm². All measurements were added together and the total expressed as a percentage of the total area analyzed.

FIG. 7 is a series of ten photomicrographs of HDNF cell migration across a fibronectin-coated surface in the absence or presence of 0.1 μg/ml excretions/secretions.

FIG. 8 is a graph that quantifies HDNF cell migration into the free space created by the cloning cylinder, as represented by percentage of cell surface area coverage of a defined area located outside of the original cell boundary. Cell migration in the absence of ES or while exposed to 0.1 μg/mL ES is presented.

FIG. 9 is a graph that quantifies ATP concentration of HDNF seeded upon a fibronectin-coated surface in the absence or presence of the specified concentration of larval ES. Results represent the mean from three replicate samples+/−one standard deviation.

FIGS. 10A and 10B are graphs providing a protease analysis of ES using several classes of protease inhibitors. FIG. 10A quantifies the hydrolysis of H-Leu-AMC substrate by STI and STI/IA-treated ES. FIG. 10B quantifies the activity of commercially available preparations of trypsin and chymotrypsin. Where specified, hydrolysis was performed in the presence of 1 mM phenylmethylsulfonyl fluoride (PMSF), 100 mM 4-(amidinophenyl)methanesulfonyl fluoride (APMSF), 5 mM L-trans-epoxysuccinyl-leucylamide-(4-guanidino)-butane (E64), 10 mM 1,10 phenanthroline, or 1 percent ethanol. Percentage activity relates to FIG. 10A residual activity relative to noninhibited untreated ES or in FIG. 10B where appropriate, noninhibited trypsin or chymotrypsin.

FIG. 11 is a series of 30 photomicrographs of HDNF cell migration across a fibronectin-coated surface in the presence of PBS or EJ that were untreated or treated with STI or STI and IA.

FIG. 12 is a graph that quantifies HDNF cell migration into the free space created by the cloning cylinder, as represented by percentage cell surface area coverage of a defined area located outside of the original cell boundary. Cells exposed to 0.1 μg/mL ES or an equivalent volume of PBS that had been left untreated or treated with immobilized STI or Immobilized STI and insolubilized IA.

FIG. 13 shows the sodium dodecyl sulfate (SDS)-polyacrylamide gel electrophoresis of fibronectin following incubation at 37° C. in the presence of various ES preparations. Lane 1 contains 0.1 μg/mL untreated ES; Lane 2 contains STI-treated ES and Lane 3 contains STI/IA-treated ES. Incubation time is as indicated. Molecular weights (kDa) are indicated at the side of each gel representing the molecular weights of the peptide bands resulting from fibronectin digestion by untreated ES. Grey arrowheads indicate peptide bands resulting from limited fibronectin digestion by STI- and STI/IA-treated ES. Following twelve hours of incubation, moving from top to bottom, the arrowheads indicate bands of 171 kDa, 105 kDa, 32 kDa, and 31 kDa. Following forty-eight hours of incubation, the arrow indicates a band of 79 kDa. The lane labeled FN represents the typical appearance of fibronectin following incubation at 37° C. in the absence of ES. Incubation time had no effect upon the fibronectin molecule incubated alone.

FIG. 14. shows the results of gel filtration of L. sericata ES products using Sephacryl S300.

FIG. 15 shows the purification of trypsin by affinity chromatography on p-aminobenzamidine agarose.

FIG. 16. illustrates substrate specificity and inhibitor profile of the L. sericata recombinant trypsin. Activated L. sericata trypsin demonstrated typical trypsin activity cleaving AMC from the positively charged arginine residue in the trypsin substrate Tosyl-Gly-Pro-Arg-AMC (A). Recombinant chymotrypsin requires an amino acid with an aromatic side chain in this position and preferentially cleaves AMC from the substrate Suc-Ala-Ala-Pro-Phe-AMC (SEQ ID NO:7). Recombinant trypsin was inhibited by the trypsin inhibitor APMSF in the range 0.3-300 μM in contrast to the recombinant chymotrypsin which remained resistant (B). Tryptic activity resolved on SDS-PAGE at approximately 26 kDa (C). ↑=Indicates substrate cleavage position, In=Inactivated enzyme, Act=activated enzyme.

FIG. 17 illustrates the degradation of eschar from venous leg ulcers by L. sericata trypsins purified by a combination of S-300 gel filtration and affinity chromatography on p-aminobenzamidine agarose.

FIG. 18 illustrates the degradation of slough/eschar by L. sericata recombinant trypsin; where Lane 1) is untreated slough/eschar; Lane 2) is activation buffer; Lane 3) contained Slough/eschar plus inactivated trypsin and Lane 4) contained Slough/eschar plus activated trypsin

### DETAILED DESCRIPTION OF THE INVENTION

The protein of the present invention exists, in nature, in the excretory/secretory (ES) secretions of the larvae of Lucilia sericata.

The larval ES secretions demonstrate a classical pH optimum of 8.0-8.5 when hydrolysing the fluorescent protein substrate fluorescein isothiocyanate-casein (FITC-casein). By pre-incubating the larval ES secretions, prior to monitoring the hydrolysis of FITC-casein, with the irreversible low molecular weight inhibitors 4-(amidinophenyl) methane sulphonyl fluoride (APMSF; an inhibitor for all trypsin-like serine proteases but not chymotrypsin-like serine proteinases) or with phenyl methanesulphonyl fluoride (PMSF; an inhibitor for all serine proteinases) it is shown that larval ES secretions have two types of serine proteinase activity: a trypsin-like activity and a chymotrypsin-like activity. The dual activity is confirmed by monitoring the hydrolysis of the fluorescent peptide substrates Tosyl-Gly-Pro-Arg-AMC (selective for trypsin-like proteinases) and Suc-Ala-Ala-Pro-Phe-AMC (SEQ ID NO: 7) (selective for chymotrypsin-like proteinases), in which “AMC” represents 7-amino-4-methyl coumarin and “Suc” represents succinyl.

In addition to the predominant serine proteinase activity detected in the ES secretions of Lucilia sericata other less predominant activity is present. The presence of an aspartyl and metalloproteinase activity has been detected though no cysteinyl activity is shown. The aspartyl activity, shown by monitoring FITC-casein hydrolysis, is pronounced at pH 5.0 and is successfully inhibited by the class specific inhibitor pepstatin A. The metalloproteinase activity present is demonstrated by the ability of the ES secretions to hydrolyse a leucine aminopeptide, revealing the presence of an exopeptidase. Exopeptidases recognise free —NH2 aminoacids in peptides. Leucine aminopeptide hydrolysis by Lucilia sericata ES is only inhibited by the Zn²⁺ chelator 1,10-phenanthroline, a classic metalloproteinsase inhibitor. This inhibition reflects the presence of an exopeptidase with a metalloproteinase enzymic nature.

The ES secretions have an α-amylase activity calculated to be about 0.88 units/litre. Additionally, phosphatase activity (hydrolysis of orthophosphoric monoester bond) is present in the larval ES secretions although this activity is approximately 50 times lower when compared to the proteinases. Lipase activity (hydrolysis of ester bonds found in fatty acid esters) is also identified. This lipase activity is not detected when the ES secretions are pre-incubated with the inhibitor PMSF, indicating that this hydrolysis is due to the serine proteinase in the secretions.

It can be concluded from our investigations that the predominant class of activity in the larval ES secretions is serine proteinase activity and that there are two types of serine proteinase activity present; one derived from a chymotryptic enzyme and one derived from a tryptic enzyme.

The protein of the present invention, which relates to the tryptic enzyme, may be obtained in substantially pure form from the crude ES secretions by a chromatographic procedure. The ES secretions are collected from the larvae of Lucilia sericata and are subjected to affinity chromatography using immobilised aminobenzamidine. Aminobenzamidine is a reversible inhibitor of trypsin-like serine proteinases. After collection of the “flow-through” material from the chromatographic procedure, i.e., the material which is not bound by the immobilised reagent, the enzyme which has been bound by the immobilised reagent may be eluted by the addition of free aminobenzamidine and collected separately.

Inhibitor studies carried out on the unbound (“flow-through”) fraction and the eluted fraction using the inhibitors APMSF and PMSF show that the unbound material has activity characteristic of chymotryptic enzymes whereas the eluted fraction is a tryptic serine proteinase.

Without being bound to a particular theory, the tryptic serine proteinase, isolated from the crude larval ES secretions as described above, has the ability to initiate and promote healing events in wounds. It has been found that larval ES secretions are capable of degrading the extra cellular matrix (ECM)/wound components, fibronectin, laminin and collagens I, III, IV and V. These macromolecules are found in the slough of chronic wounds and also make up the “fibrin cuffs” that are predominant in chronic ulcers. The degradation of laminin and fibronectin by larval secretions is inhibited by PMSF, but not significantly by APMSF or by the metalloproteinase inhibitor 1,10-phenanthroline.

In addition, the degradation of the different forms of collagen by the predominant serine proteinase activity indicates the presence of a collagenase. This suggests that the serine proteinase activity within larval ES secretions has the potential to degrade key wound components, a requirement for the debridement stage of healing, and is involved in preventing “fibrin cuff” formation, stimulating angiogenesis, leucocyte invasion and fibroplast proliferation. In contrast to the ECM components degraded, no degradation of fibrin itself, elastin and hyaluronic acid by ES secretions has been detected, suggesting the absence of plasmin or elastase-like proteinase or hyaluronidase.

The trypsin-like enzyme isolated from crude larval ES secretions, we believe, has the ability to activate healing processes via protease-activated receptors (PARs). PAR1, which is known to be cleaved by thrombin, trypsin and factor Xa, is present on human endothelial cells, fibroblasts, keratinocytes, platelets, monocytes and smooth muscle cells. PAR1 agonists promote endothelium dependent vasodilation, leading to extravasation of plasma proteins and leucocytes, to cell mitogenesis, and to the healing of inflamed and damaged tissue. The PARs have attached ligands which are cleaved proteolytically by the trypsin-like enzyme of the invention to release peptides which then act to trigger other biochemical processes involved in promoting wound healing. The arginine residue at the cleavage site of the tethered ligand of PAR1 is susceptible to the action of trypsin-like proteinases. Further details of the tethered ligands and the elucidation of their sequence is given in Am. J. Physiol. 274 (1998), c1428-c1452.

As mentioned above, PARs have tethered ligands which when cleaved by the trypsin-like serine proteinase release one or more peptides into the wound area.

Normally tethered ligands of PARs which are believed, by us, to be responsible for the activation of wound healing when released include the peptides 1 to 4 below

| | |
|---|---|
| 1. Ser-Phe-Leu-Leu-Arg-Asn; | (SEQ ID NO: 3) |
| 2. Ser-Leu-Ile-Gly-Lys-Val; | (SEQ ID NO: 4) |
| 3. Thr-Phe-Arg-Gly-Ala-Pro; | (SEQ ID NO: 5) |
| and | |
| 4. Gly-Tyr-Pro-Gly-Gln-Val. | (SEQ ID NO: 6) |

These four peptides are agonists of PARs 1 to 4, respectively.

Without being bound to a particular theory, wound healing is promoted by the application to the wound area of one or more of these peptides, or a protected analogue thereof which is protected against aminopeptidase activity, without the need to apply the tryptic serine proteinase to the wound.

The peptides of the invention, as described above, can be prepared synthetically and purified according to the usual routes of peptide synthesis and purification known in the art. The peptide may be protected against aminopeptidase activity to enhance activity and/or to prolong the period within which the peptide remains active in the wound area. Protection against aminopeptidase activity may, for example, be achieved by the amidation at COOH substitution in the peptide using a non-coded anomalous amino acid and/or CO—NH amide bond replacement by an isostere.

The peptides (and/or protein) of the invention may be applied to a wound to induce a profile of growth factors conducive to healing. For instance, one or more peptides, either in a pure form or in a sterile carrier, can be sprinkled over the wound area or incorporated into a carrier to be applied to the wound. For instance, the peptide (and/or protein) can be incorporated or encapsulated into a suitable material capable of delivering the peptide to a wound in a slow release or controlled release manner. An example of such a suitable material is poly(lactide-co-glycolide) or PLGA particles which may be formulated to release peptides in a controlled release manner. Alternatively, one or more peptides (and/or protein) may be incorporated into a dressing to be applied over the wound. Examples of such dressings include staged or layered dressings incorporating slow-release hydrocolloid particles containing the wound healing material or sponges containing the wound healing material optionally overlayered by conventional dressings. Hydrocolloid dressings of the type currently in use, for example those available under the trademark “Granuflex™”, may be modified to release the peptides to the wound.

As mentioned above, the invention also relates to functional homologues of the Lucilia protein of the invention which may be obtained from other insects having larval phases and which can potentially be used in the treatment of wounds. For example, given the teaching of the present application, it would be within the ability of the skilled person, using the experimental methods disclosed herein, to assay larval secretions from other non-Lucilia organisms for a functional homologue of the Lucilia protein of the invention. Furthermore, proteinases according to the invention produced by Lucilia which promote wound healing may be cloned and expressed in bulk in an active recombinant form in an insect/baculovirus expression system. Genes corresponding to the proteinases will be identified by PCR or immunological screening from appropriate cDNA libraries and manipulated for expression in this system. As proteinases from diverse sources can prove difficult to express in prokaryotic expression systems, it is advantageous to use an insect expression system for the expression of the insect gene corresponding to the proteinase of the invention.

In another embodiment, the invention features compositions and methods that are useful for wound healing. The invention is based, at least in part, on the discovery that L. sericata excretions or secretions (ES) alter fibroblast migration in an in vitro wound assay. In particular, it was discovered that serine protease activities within ES (e.g., serine proteinase activities and metalloproteinase activity) promoted fibroblast migration across a fibronectin-coated surface by degrading fibronectin. Accordingly, the invention provides compositions and methods that promote wound healing featuring an isolated L. sericata polypeptide having serine protease activity that hydrolyses Tosyl-Gly-Pro-Arg-AMC.Hcl substrate, where the protease activity is reduced following incubation with soybean trypsin inhibitor or iminodiacetic acid.

Desirably, the ES serine protease degrades fibronectin. The invention further provides biologically active fragments of fibronectin that promote wound healing (e.g., 182 kDa to 29 kDa, including fibronectin bands of 22, 31, 32, 40, 79, 105, and 171 kDa in size) that are the product of incubation with ES. The invention also provides therapeutic compositions comprising a metalloproteinase derived from L. sericata ES that degrades an H-Leu-AMC substrate, where the metalloprotease activity is reduced following incubation with 1,10 phenanthroline.

### Maggots and Wound Healing

In the procedure termed “biosurgery,” Lucilia sericata larvae (Diptera: Calliphoridae) are applied to necrotic and sloughy wounds that have not responded to conventional methods of debridement. Through clinical observations, biosurgery is recognized to debride the wound of necrotic tissue, aid wound disinfection, and promote granulation tissue formation (Mumcuoglu, Am J Clin Dermatol 2001; 2:219-27; Sherman, Annu Rev Entomol 2000; 45:55-81; Thomas, J Wound Care 1996; 5:60-9; Wolff Acta Derm-Venereol 1999; 79:320-1; Wollina et al., Int J Dermatol 2002; 41:635-9).

Various L. sericata larval extracts and secretions increase the proliferation of human fibroblasts in vitro, but cell proliferation is only one aspect of granulation tissue formation. In addition, fibroblasts orchestrate fibroplasia and lead granulation tissue migration into the fibrin clot. Interactions between fibroblasts and the surrounding extracellular matrix (ECM) play a crucial role in tissue formation. For example, through binding to cell membrane receptors, individual ECM components, such as fibronectin and collagen, provide a scaffold for contact guidance, controlling fibroblast adhesion and facilitating directional cell migration.

Proteases derived from many sources, including fibroblasts, modulate such interactions by degrading the ECM and by interacting with cell surface receptors such as the urokinase plasminogen activator receptor (Mignatti et al., In: Clark RAF, editor. The molecular and cellular biology of wound repair, 2nd Ed. New York: Plenum Press, 1996:427-74) or the protease-activated receptors (PARs) (De'ry et al., Biochem Soc Trans 1999). Consequences of these interactions include the induction of fibroblast proliferation (De'ry et al., Biochem Soc Trans 1999; 27:246-54; Abe et al., J Allergy Clin Immunol 2000; 106 (Suppl.):78-84) and the promotion of angiogenesis (Blair et al., Clin Invest 1997; 99:2691-7). Proteases may also exert indirect effects through the release of proteolytic breakdown products of ECM components, such as fibronectin. These bioactive peptides have been associated with inducing fibroblast migration and chemotaxis, reepithelialization, and tissue remodeling. Recent studies have shown L. sericata larval secretions to be rich in proteolytic activity. These secretions, containing serine proteinase, aspartyl proteinase, and metalloproteinase activities, more properly termed excretions/secretions (ES) because it is not known by which method(s) these substances are released, have been shown to degrade common ECM components that are present within the wound. These substrate proteins include fibrin clots, fibronectin, collagen, and laminin (Chambers et al., Br J Dermatol 2003; 148:14-23; Horobin et al., Br J Dermatol 2003; 148:923-33). The proteolytic activity of ES has also been associated with modifying human, dermal fibroblast adhesion to collagen- and particularly fibronectin-coated surfaces (Horobin et al., Br J Dermatol 2003; 148:923-33).

Polypeptides Isolated from Lucilia sericata and Derivatives Thereof.

Lucilia sericata secretions or excretions are known to have significant wound healing activity. As reported herein, this activity is due, at least in part, to the presence of serine proteases that degrade fibronectin and enhance fibroblast migration to promote wound healing. Accordingly, the invention provides compositions comprising at least one polypeptide or biologically active fragment thereof having serine protease activity or fibronectin degrading activity, fibroblast migration promoting activity, or wound healing activity, where the polypeptide is isolated from L. sericata (e.g., an L. sericata secretion or excretion). Methods for assaying ES for proteolytic activity are described herein, and include, for example, methods for analyzing the release of 7-amino-4-methylcoumarin (AMC) from a synthetic peptide substrate, such as Tosyl-Gly-Pro-Arg-AMC.HCl, Suc-Ala-Ala-Pro-Phe-AMC (SEQ ID NO:7), Z-Phe-Arg-AMC.HCl, H-Pro-Phe-Arg AMC.2HCl, Z-Gly-Gly-Arg-AMC.HCl, H-Leu-AMC, and H-Arg-AMC.2HCl. Typically a polypeptide of the invention will be a naturally produced protein secreted by an insect in its larval stage. Suitable insects are envisaged as being those that are used in their larval form in the treatment of wounds.

In other embodiments, the invention provides biologically active fragments of fibronectin that promote wound healing, wherein the peptide fragments have a size between 182 kDa to 29 kDa. In particular, the invention provides biologically active fragments of fibronectin that promote wound healing or enhance fibroblast migration or motility and that comprise at least about 22, 31, 32, 40, 79, 105, and 171 kDa in size as measured by SDS-electrophoresis as described herein in the Examples.

Compositions of the invention include naturally derived compounds having a variety of biological activities that promote wound healing, that degrade fibronectin (e.g., into or that increase fibroblast migration or motility that are isolated from a larval ES or larval extract or are isolated from fibronectin) using methods known in the art. Briefly, such extracts are typically prepared by solubilizing ES in an appropriate solvent, or by immersing a pulverized larva (or a part of a larva) in an aqueous solvent, an organic solvent, or a mixture of solvents. Exemplary organic solvents include ethanol, dichloromethane, and hexane. The crude extract thus obtained can be filtered or centrifuged to remove insoluble materials. A purified extract is then obtained from the crude extract using liquid chromatography (e.g., high-pressure liquid chromatography) or other suitable methods. An extract can be produced either by a batch method or by a continuous method. Such extracts are then screened for biological activity. Active fractions are then isolated using routine methods.

Polypeptides of the invention include the naturally derived compounds themselves and their derivatives (e.g., sugar derivatives, metabolic derivatives, prodrugs, derivatives by isomerization, oxidization product, and reduction product). Such derivatives may be naturally occurring or synthetic derivatives. A compound that is chemically derived may be obtained through a natural biologic process, such as living organism metabolism (e.g., an insect, mammalian, plant, or bacterial metabolic process) or may be obtained through synthetic processes using human intervention (e.g., chemical synthesis) from a biological extract (e.g., larval or extracellular matrix extract). The peptides of the invention, as described above, can be prepared synthetically and purified according to the usual routes of peptide synthesis and purification known in the art. The peptide may be protected against aminopeptidase activity to enhance activity and/or to prolong the period within which the peptide remains active in the wound area. Protection against aminopeptidase activity may, for example, be achieved by the amidation at COOH substitution in the peptide using a non-coded anomalous amino acid and/or CO—NH amide bond replacement by an isostere.

A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention.

### Screening Assays

Methods of screening for wound healing activity are known in the art and are described herein. In one embodiment, an isolated L. sericata polypeptide, a fibronectin degradation product (e.g., produced by incubating fibronectin together with ES), or any other polypeptide of the invention or fragment thereof is assayed for an effect on cell migration using any conventional method known in the art. In one embodiment, cell migration is assayed using a two-dimensional in vitro wound system. In one embodiment of this assay, the cells are skin cells, such as fibroblasts, keratinocytes, or endothelial cells. Comparable migration assays are also useful in the methods of the invention and are well known to the skilled person and comprise, for example, the Boyden chamber method, the scratch assay, the colloidal gold assay and an assay based on the migration in a fibrin matrix. In a scratch assay, cells are seeded on a tissue culture plate and are grown to confluency. The confluent cell layer is then wounded under standard conditions with a plastic pipet tip to create a cellfree zone. Subsequently, test substances can be added after and migration into the cellfree zone can be monitored by photo documentation of identical locations in the scratch. For the colloidal gold assay, coverslips are coated with colloidal gold salts and covered with a suitable substratum, for example Collagen I. Cells, for example keratinocytes or fibroblasts are plated on the cover slip and allowed to migrate for several hours. Afterwards the cells are fixed in formaldehyde and migration tracks can be analysed using computer assisted image analysis. In the assay based on the migration in a fibrin matrix, cells are plated onto a fibrin matrix, that is obtained from freeze-dried surgical fibrinogen and distributed onto culture dishes before clotting. The fibrin matrix is transparent and therefore suitable for microscopic analysis of the cells. Suitable cells, for example fibroblasts or keratinocytes are incubated on the matrix for 24 hours, fixed with formaldehyde and tunnels generated by migrating cells in the matrix are examined by light microscopy. In other embodiments, the methods of the invention assay cell proliferation as described herein, by monitoring ATP in a luciferase assay. Other suitable assay systems for measuring cell proliferation are known in the art and include, for example, the incorporation of labeled nucleotides into the DNA of the cells (see, e.g., Savino and Dardenne, 1985, J. Immunol. Methods 85: 221-6; Perros and Weightman, 1991, Cell Prolif. 24: 517-23; de Fries and Mitsuhashi, 1995, J. Clin. Lab. Anal. 9: 89-95) by staining the cells with specific dyes (Schulz et al., 1994, J. Immunol. Methods 167: 1-13) or by means of immunological methods (Frahm et al., 1998, J. Immunol. Methods 211: 43-50).

Quantitatively defined amounts of prepared ES, L. sericata polypeptides, or fibronectin fragments of the invention can be tested for functionality by bringing the polypeptide(s) to be tested into contact with a suitable cell type (e.g., fibroblasts, keratinocytes). Migration of the cells is quantified and then compared with the expected, standard migration value. Only preparations that increase migration relative to the expected standardized effect on migration are identified as useful in a therapeutic or diagnostic agent of the invention.

Polypeptides of the invention identified as useful in an in vitro assay may be tested, if desired, in an in vivo assay system carried out in mice, for example, to determine whether the application of a polypeptide of invention to a wound alters the healing of the wound. This can be done, for example, by measuring the rate of reepithelialization.

The compounds of this invention may be modified by appending appropriate functionalities to enhance selective biological properties. Such modifications are known in the art and include those which promote wound healing. A compound that is chemically derived may be obtained through a natural biologic process, such as living organism metabolism or may be obtained through synthetic processes using human intervention (e.g., chemical synthesis) from a larval secretion, excretion, or extract.

Compounds useful in the combinations of the invention often possess multiple biological activities that promote wound healing. Such biological activities include serine proteinase activity, metalloproteinase activity, fibronectin degrading activity, or fibroblast migration promoting activity. Accordingly, the description of a particular compound as having a particular activity is not intended to limit the invention.

Compounds identified using the methods described herein are useful treating a chronic wound, or for a related disease and/or disorder or symptom thereof. Such methods comprise administering a therapeutically effective amount of a pharmaceutical composition comprising a compound of the formulae herein to a subject (e.g., a mammal such as a human). Thus, one embodiment is a method of treating a subject suffering from or susceptible to the formation of a chronic wound, or a related disease or disorder or symptom thereof. The method includes the step of administering to the mammal a therapeutic amount of an amount of a compound herein sufficient to treat the disease or disorder or symptom thereof, under conditions such that the disease or disorder is treated. Identifying a subject in need of such treatment can be in the judgment of a subject or a health care professional and can be subjective (e.g. opinion) or objective (e.g. measurable by a test or diagnostic method).

### Formulation of Pharmaceutical Compositions

Polypeptides of the invention, including isolated polypeptides that are naturally present in an ES derived from L. sericata, or fragments thereof, as well as fragments of a fibronectin polypeptide (e.g., fragments that are released when an intact fibronectin polypeptide is incubated with ES) are useful for promoting wound healing.

Accordingly, the compositions of the present invention are formulated for topical administration. Suitable formulations include gels, sprays, ointments, and creams. Administration of each compound of the combination may be by any suitable means that results in a concentration of the compound that, combined with the other compound, is effective. Each compound can be admixed with a suitable carrier substance, and is generally present in an amount of 1-95% by weight of the total weight of the composition. If desirable, the compounds can be formulated together.

The pharmaceutical compositions may be formulated for topical use according to conventional pharmaceutical practice (see, e.g., Remington: The Science and Practice of Pharmacy, (20th ed.) ed. A. R. Gennaro, 2000, Lippencott Williams & Wilkens, Philadelphia, Pa., and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time period after administration, using controlled release topical formulations.

Therapeutic compositions suitable for topical application include conventional anhydrous or aqueous preparations including ointments, lotions, creams, pastes, jellies, sprays, aerosols, and oils. These preparations can include oleaginous, aqueous, or, emulsion-type bases. Optionally, topically applied formulations can be covered with an occlusive or semi-occlusive dressing.

### Means for Delivery

Compositions of the invention comprising a polypeptide of the invention (e.g., a biologically active fibronectin fragment or L. sericata polypeptide having serine protease activity fibronectin degrading activity, fibroblast migration promoting activity, or wound healing activity) are provided in any conventional manner useful for the application of therapeutics to a wound. Such means include dressings, plasters, compresses or gels which contain compounds according to the invention. Thus, it is possible to administer drugs comprising suitable additives or auxiliary substances, such as physiological sodium chloride solution, demineralized water, stabilizer, proteinase inhibitors, gel formulations, such as white vaseline, low-viscosity paraffin and/or yellow wax, etc., topically and locally in order to exert an immediate and direct effect on the wound healing process. The topical administration of therapeutic compositions can be effected, for example, in the form of a cream, a foam, an aerosol spray, an injection, a gel matrix or a sponge or in the form of drops or washings. The treatment can also be effected using a transdermal therapeutic system which provides for drug release in a chronologically controlled manner.

In other embodiments, a polypeptide of the invention is delivered to a wound using a polymeric material to form a delivery system. Preferably, the polymer contains an effective amount of a polypeptide of the invention. An adhesive or other adhering means may be applied to the outer edges of the polymeric material to hold the patch in position during the delivery of the chemical or pharmaceutically active component. This polymeric delivery system provides for the systematic and/or locally administration of a desired amount of a therapeutic agent.

Other embodiments of the present invention include wound-healing devices configured and produced as a woven sheets. Such sheets provide a means for temporarily treating and sealing an open wound. Additionally, the polypeptides of the invention may be provided in combination with any other pharmacologically active agents that promote the healing of the tissue within and around the wound.

The peptides (and/or protein) of the invention may be applied to a wound to induce a profile of growth factors conducive to healing. For example, one or more peptides, either in a pure form or in a sterile carrier, can be sprinkled over the wound area or incorporated into a carrier to be applied to the wound. For instance, the peptide (and/or protein) can be incorporated or encapsulated into a suitable material capable of delivering the peptide to a wound in a slow release or controlled release manner. An example of such a suitable material is poly(lactide-co-glycolide) or PLGA particles which may be formulated to release peptides in a controlled release manner. Alternatively, one or more peptides (and/or protein) may be incorporated into a dressing to be applied over the wound. Examples of such dressings include staged or layered dressings incorporating slow-release hydrocolloid particles containing the wound healing material or sponges containing the wound healing material optionally overlayered by conventional dressings. Hydrocolloid dressings of the type currently in use, for example those available under the trademark “Granuflex”, may be modified to release the peptides to the wound.

In still other embodiments, a polypeptide of the invention is administered directly to an injured area, for example, by sprinkling, packing, implanting, inserting or applying or by any other administration means to open wounds on the body.

### Dosages

Therapeutic compounds of the invention (e.g., polypeptides, and biologically active fragments thereof described herein) are administered in an effective amount. The dosage of each compound used in any given therapeutic method depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used. In general, the compositions of the invention are administered topically. As described herein, compositions of the invention, including isolated ES secretions, isolated polypeptides, analogs, and functional fragments thereof are administered in a solution in a range between at least about 0.01 to 100 μg/ml (e.g., 0.01, 0.05, 0.1, 0.25, 0.5, 0.75, and 1.0 μg/ml). In particular embodiments, compositions are administered in solutions of 0.1, 0.25, or 0.5 μg/ml. In certain embodiments, compounds of the invention, such as those described herein, formulated in compositions suitable for topical administration where the dosage levels range between at least about 0.0001 to 4.0 grams once per day (or multiple doses per day in divided doses) for adults. Thus, in certain embodiments of this invention, a compound herein is administered at a dosage of any dosage range in which the low end of the range is any amount between 0.1 μg/day and 400 mg/day and the upper end of the range is any amount between 1 μg/day and 4000 mg/day (e.g., 0.1 μg/day, 0.5 μg/day, 1 μg/day, 5 μg/day, 10 μg/day, 25 μg/day, 50 μg/day, 100 μg/day, 5 mg/day and 100 mg/day, 150 mg/day and 500 mg/day, 300 mg/day-1000 mg/d (topical)). In other embodiments, a compound herein, is administered at a dosage range in which the low end of the range is any amount between 0.1 mg/kg/day and 90 mg/kg/day and the upper end of the range is any amount between 1 mg/kg/day and 100 mg/kg/day (e.g., 0.5 mg/kg/day and 2 mg/kg/day, 5 mg/kg/day and 20 mg/kg/day). In other embodiments, a combination of the invention is administered at a dosage of 1.5 mg/kg/day, 15 mg/kg/day, 30 mg/kg/day. The dosing interval can be adjusted according to the needs of individual patients. For longer intervals of administration, extended release or depot formulations can be used. It has been found that 1 unit of trypsin of SEQ ID NO: 6 will generally treat 50 units of tissue, and thus 1 mg of trypsin will treat 50 mg of wound tissue.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which defects in wound healing may be implicated.

### Kits or Pharmaceutical Systems

The present compositions may be assembled into kits or pharmaceutical systems for use in ameliorating a wound or promoting wound healing. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the compounds of the invention. In various embodiments, such kits are labelled for use in wound treatment or include directions for the use of the polypeptides of the invention to promote wound healing.

### Polypeptide Expression

The invention further provides recombinant polypeptides that correspond to the naturally derived polypeptides described herein (e.g., L. sericata polypeptides and biologically active fragments thereof, as well as fragments of fibronectin produced by incubating a fibronectin polypeptide with ES). Methods for identifying the nucleic acid sequences encoding such polypeptides are known in the art. In general, polypeptides of the invention are produced by transformation of a suitable host cell with all or part of a polypeptide-encoding nucleic acid molecule or fragment thereof in a suitable expression vehicle. Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains which express T7 RNA polymerase in response to IPTG induction. Once produced, a recombinant polypeptide is then isolated according to standard methods known in the art.

Another bacterial expression system for polypeptide production is the pGEX expression system (Pharmacia). This system employs a GST gene fusion system which is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3* may be cleaved with factor Xa.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). Also included in the invention are polypeptides which are modified in ways which do not abolish their biological activity (assayed, for example as described herein). Such changes may include certain mutations, deletions, insertions, or post-translational modifications, or may involve the inclusion of any of the polypeptides of the invention as one component of a larger fusion protein.

The invention further includes analogs of any naturally occurring polypeptide of the invention. Analogs can differ from the naturally occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino acid sequence of the invention. The length of sequence comparison is at least 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β- or γ-amino acids.

In addition to full-length polypeptides, the invention also includes fragments of any one of the polypeptides of the invention. As used herein, the term “fragment,” means at least 5, preferably at least 20 contiguous amino acids, preferably at least 30 contiguous amino acids, more preferably at least 50 contiguous amino acids, and most preferably at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events). The aforementioned general techniques of polypeptide expression and purification can also be used to produce and isolate useful peptide fragments or analogs (described herein).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The invention is not limited to the embodiments hereinbefore described which may be varied without departing from the invention.

Other features and advantages of the invention will be apparent from the detailed description, and from the claims.

### EXAMPLES

### Example 1

### Isolation and Assay of the Trypsin-Like Serine Proteinase of the Invention

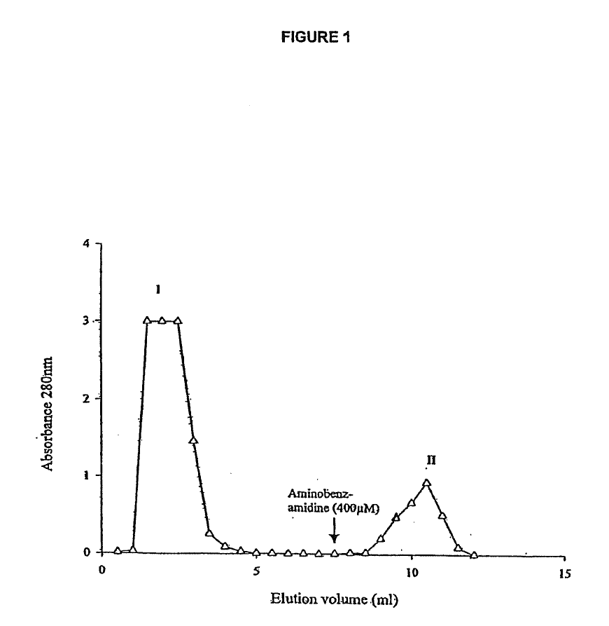

The trypsin-like serine proteinase was purified by affinity chromatography of Lucilia sericata ES on aminobenzamidine agarose. The column matrix (1 ml) was equilibrated with 20 ml of 0.025M Tris-HCl buffer pH 8.0 containing 0.5M NaCl. The crude ES (0.5 ml, 70 μg/ml protein) was diluted with an equal volume of buffer before application to the column. Fractions (0.5 ml) were collected throughout the chromatography. After washing with 6.5 times column volume of buffer to remove unbound protein, the free aminobenzamidine ligand (2 ml 400 μM) was used to elicit the elution of bound material. Absorbance readings of the fractions at 280 nm were used to establish the positions of the unbound (flow-through) and bound peaks which were then collected for assay. The elution profile is shown in FIG. 1

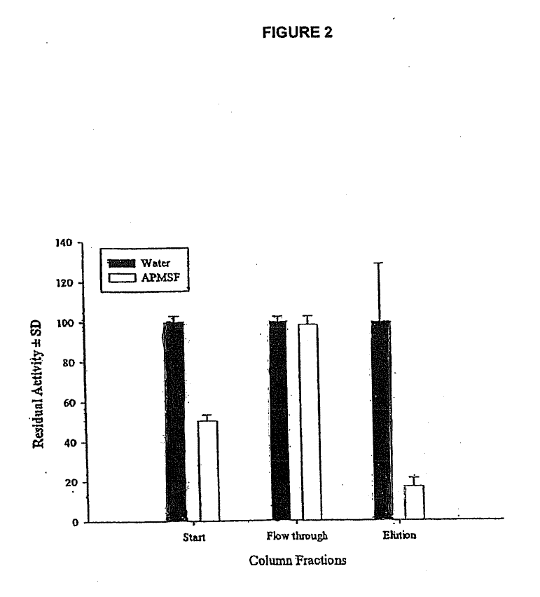

Aminobenzamidine agarose binds trypsin-like serine proteinases. Following application of larval enzyme secretions to the column, unbound material passed directly through and was collected as “flow-through” (peak I). The addition of free aminobenzamidine to the column buffer elicited elution of the bound proteinase (peak II). The unbound (flow-through) material contained proteinase activity unaffacted by APMSF (possibly including a chrymotrypsin-like enzyme), whereas the activity in the aminobenzamidine elution peak was substantially abolished (80%) by APMSF, indicating purification of a trypsin-like serine proteinase activity. The residual activities of the column fractions are shown in FIG. 2.

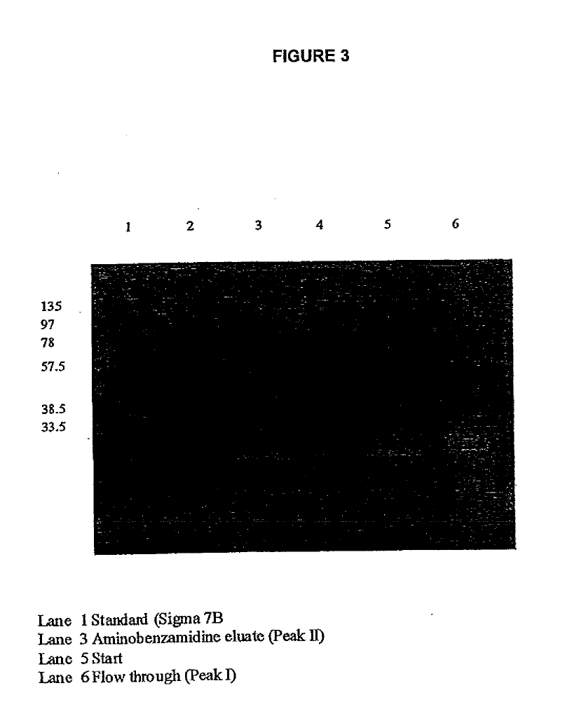

Column fractions were examined by electrophoresis in non-reducing SDS sample buffer (0.5M Tris-HCl pH 6.8 containing 4% SDS, 20% glycerol and 0.02% bromophenol blue) on 12% SDS polyacrylamide gels containing 0.1% human haemoglobin. SDS was removed by washing in 2.5% Triton X-100 (1 h) and distilled water (15 min). Proteolysis of the haemoglobin substrate in the gel by incubation at 37° C. in 0.1M Tris-HCl buffer pH 8.0 overnight produced clear bands revealed by protein staining in Coomassie Brilliant blue corresponding to the positions of proteinase enzymes (FIG. 3). The start and flow through fractions each showed several proteinase activities however the aminobenzamidine eluted a single band. Thus the trypsin-like enzyme previously identified in the aminobenzamidine-eluted fraction (FIG. 2) was shown to have molecular weight 25 KDa (FIG. 3).

### Example 2

### Investigation of Proteolytic Behaviour of the Larval Enzyme (ES) with FITC-Casein

The activity of Lucilia sericata ES in FITC-casein hydrolysis at pH8 was investigated using different presentations of ES (0.25 μg) as follows:

A. ES+H2O

B. ES+ethanol

C. ES pre-incubated with 0.2 mM PMSF

D. ES pre-incubated with 0.6 mM PMSF

E. ES pre-incubated with 1 mM PMSF

F. ES pre-incubated with 0.04 mM APMSF

G. ES pre-incubated with 0.12 mM APMSF

H. ES pre-incubated with 0.2 mM APMSF

The proteolytic activity of Lucilia sericata ES was inhibited following pre-incubation with the irreversible serine proteinase inhibitor PMSF. It was totally inhibited in the case where the ES had been pre-incubated with 1 mM PMSF. PMSF is dissolved in ethanol and the effect of the solvent on the activity of the ES was negligible. In contrast approximately 50% of residual serine proteinase activity from ES was detected in the cases where the ES had been pre-incubated with the irreversible “trypsin-like” specific inhibitor APMSF. Residual activity in the presence of APMSF indicates the presence of a chymotrypsin-like enzyme. The activity (%) values obtained were as follows:

A. 100%

B. 85.5%

C. 13.8%

D. 18%

E. 0%

F. 43.5%

G. 47%

H. 54%

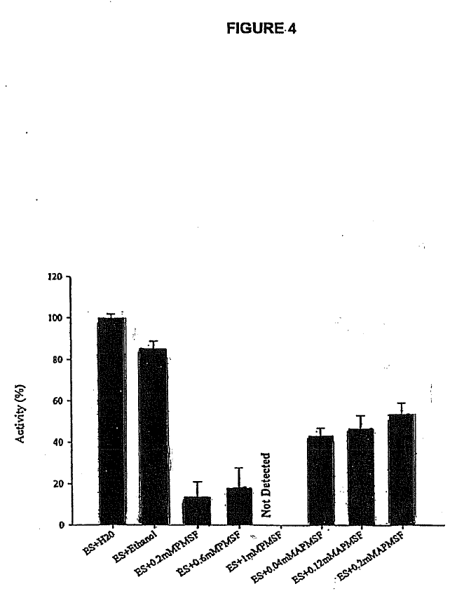

These results are shown graphically in FIG. 4.

### Example 3

### Investigation of the Proteolytic Activity of the Larval Enzyme (ES) Against Specific Substrates

The activity of Lucilia sericata ES (0.25 μg) against Tosyl-Gly-Pro-Arg-AMC (a) and against Suc-Ala-Ala-Phe-AMC (b) in the presence of APMSF and PMSF was investigated using different presentations of ES as follows: \

(a)

A. ES

B. ES pre-incubated with 0.025 mM APMSF

C. ES pre-incubated with 0.05 mM APMSF

D. ES pre-incubated with 1 mM PMSF

(b)

E. ES

F. ES pre-incubated with 0.2 mM APMSF

G. ES pre-incubated with 1 mM PMSF

The residual activity (%) values obtained were as follows:

(a)

A. 100%

B. 14.3%

C. 3.6%

D. 0%

(b)

E. 100%

F. 86.8%

G. 1.3%

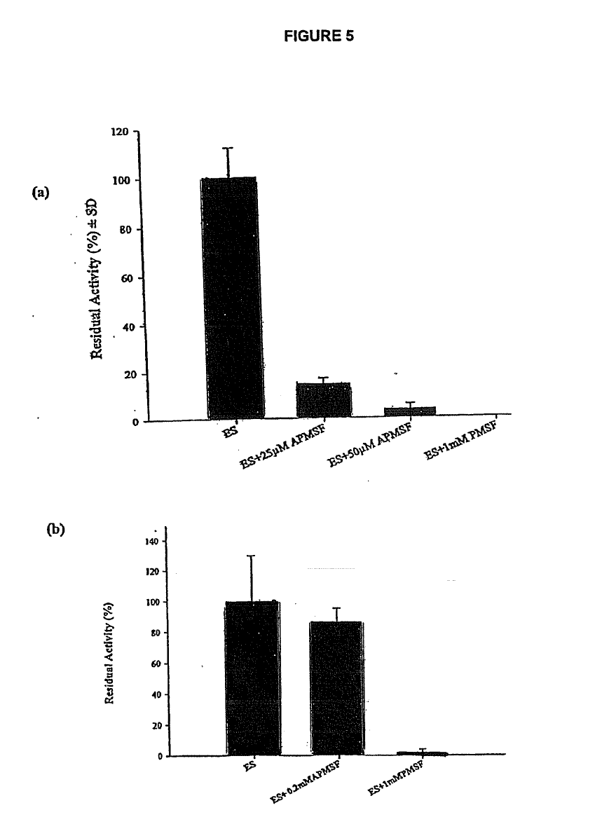

The results are shown graphically in FIG. 5.

The results for (a) reveal the “trypsin-like” serine proteinase activity present in Lucilia sericata ES. The hydrolysis of Tosyl-Glyc-Pro-Arg-AMC (selective for the serine proteinases thrombin and plasmin) was inhibited by 1 mM PMSF and 0.05 mM APMSF. However, the hydrolysis of the chymotryptic substrate Suc-Ala-Ala-Phe-AMC by Lucilia sericata ES was only inhibited by PMSF (1 mM) and not by excess APMSF (which does not inhibit chymotrypsin). The results provide further evidence of the presence in ES of two different sub-classes of serine proteinase.

### Example 4

### Cells Migrate More Rapidly in the Presence of Fibronectin

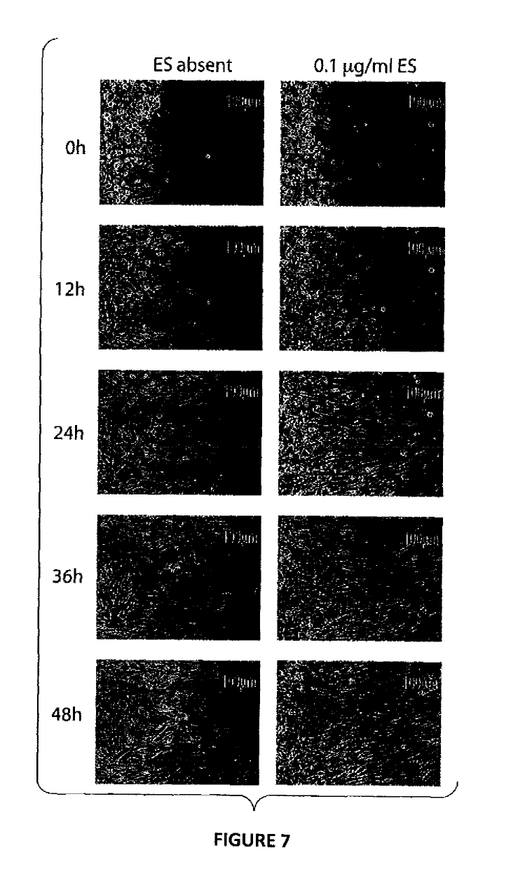

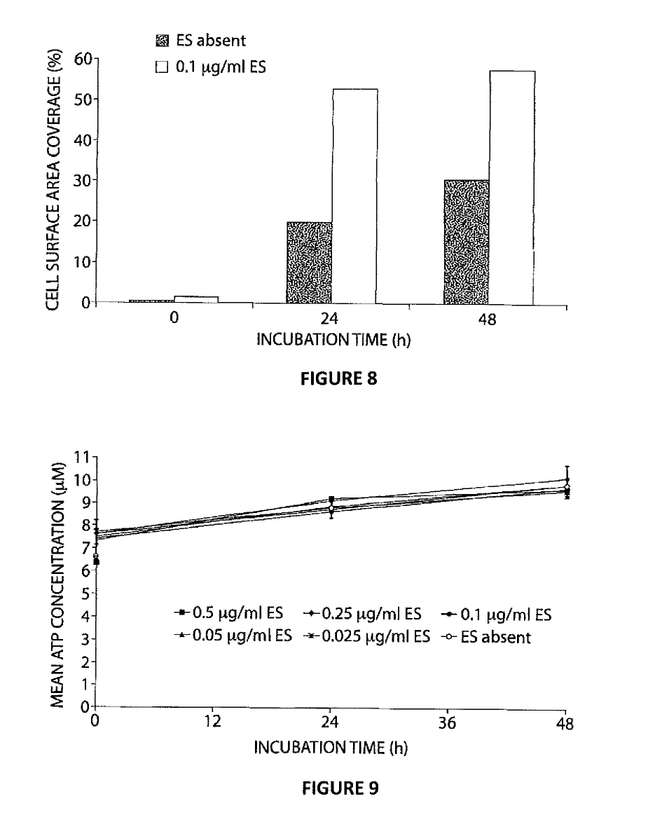

Initially, two-dimensional in vitro wound assays were assembled containing 1×10⁶ HDNF cells seeded upon surfaces coated with 100 μg/ml fibronectin. Larval excretions/secretions (ES) at a concentration of 0.1 μg/ml or an equivalent volume of PBS was also added at the time of cell seeding. For each assay, digital images monitoring the migration of HDNF cells into the cell-free area were taken over forty-eight hours and compiled into a time-lapse movie. The movies and selected stills extracted from them (FIG. 7) show that, upon seeding in serum-free conditions, the cells were able to adhere and spread upon the fibronectin-coated surface, regardless of the presence or absence of ES. Previous work found that a fibronectin coating of 1 μg/ml or greater was sufficient to facilitate fibroblast adhesion and migration where no serum proteins were present. Over the ensuing forty-eight hours, cells exposed to the PBS control or to 0.1 μg/ml ES proceeded to migrate into the free space, moving across the screen to the right-hand side of the field of view. As shown in FIG. 7 and in the relevant time-lapse movies, cells that were migrating in the presence of 0.1 μg/ml ES made more rapid progress than cells within the control. Indeed, the still images show that, by twenty-four hours, HDNF cells in the presence of ES had already reached the right-hand side of the field of view. The relevant time-lapse movie shows the cells continuing to migrate after this point, moving off-screen, further into the cell-free area created by the cloning cylinder. Quantitative analysis of images, showing the progress of cell migration after zero, twenty-four, or forty-eight hours of incubation, confirmed that cells exposed to 0.1 μg/ml ES displayed a greater rate of migration than the controls. In the presence of 0.1 μg/ml ES, cell surface area coverage had increased to 53 percent by twenty-four hours and 58 percent by forty-eight hours (FIG. 8). These values were 2.6 times and 1.9 times higher, respectively, than the coverage attained by cells in the absence of ES over the same time periods.

Observations that were made of the assay at a lower magnification, immediately after the forty-eight-hour time period had elapsed, indicated that cells exposed to 0.1 μg/ml ES had migrated at least twice as far as the field of view used in the time-lapse movie could show.

### Example 5

### ES Effect Did not Effect HDNF Proliferation

The results from these studies indicated that the presence of larval ES promoted HDNF cell migration. This is not related to an increase in cell proliferation because in the present system the progress of individual cells was visibly tracked in real time as they migrated across the culture surface. In addition, this system provides for the observation of cell division. Analysis of the time-lapse movies indicated that ES enhanced the progression of the leading cell edge by stimulating cell locomotion and not cell proliferation. The visual tracking of individual cells exposed to 0.1 μg/ml ES revealed them to be moving more rapidly than cells migrating in the absence of ES. In addition, cell division was not more frequent. To confirm these observations, an ATP assay was employed to measure the number of cells present upon a fibronectin-coated surface within medium containing the specified concentration of ES or an equivalent volume of PBS (ES absent). Results showed that concentrations of ES, ranging from 0.5 μg/ml to 0.025 μg/ml and including 0.1 μg/ml, had no effect upon ATP concentration. Thus, ES had no effect on cell number when compared with a control not treated with ES (FIG. 9). This was confirmed when the data were analyzed using two-way ANOVA. As expected, incubation time exerted a significant effect upon ATP concentration (p<0.0001) as the cells proliferated over time, although the conditions in which the cells were exposed (the absence or presence of ES at the specified concentration) did not significantly influence ATP concentration (p>0.05), nor was there a significant interaction between incubation time and sample conditions (p>0.05).

### Example 6

### Effect of Protease Inhibitors on ES Proteolytic Activity

To characterize its residual proteolytic activity, ES was treated with known proteinase inhibitors that were immobilized on solid matrices. As shown in Table 2, the untreated ES displayed activity, at pH 7.3, against all of the substrates tested except H-Arg-AMC-2HCl, indicating a lack of cathepsin (cysteine proteinase) and aminopeptidase (matrix metalloproteinase B) activity at this pH.

TABLE 2
Proteolytic activity of L. sericata larval ES, as determined by the release
of 7 AMC from enzyme class-specific substrates

| | | Percentage of untreated ES * | |
|---|---|---|---|
| | Specific activity of | | STI/ |
| | AMC released | | iminodiacetic |
| Substrate | (μM/min/mg ES) | STI-treated ES | acid treated ES |
| Tosyl-Gly-Pro- | 1226.0 +/− 21.7 | 0.5 +− 0.1 | nda |
| Arg-AMC-HCl | | | |
| H-Pro-Phe-Arg | 85.1 +− 3.2 | nda | nda |
| AMC-2HCl | | | |
| Z-Gly-Gly-Arg- | 104.9 +− 2.5 | nda | nda |
| AMC-HCl | | | |
| Z-Phe-Arg-AMC- | 66.9 +− 1.6 | 1.8 +− 0.8 | nda |
| HCl | | | |
| Z-Phe-Arg-AMC- | 43.4 +− 1.9 | — | — |
| HCl (5 mm | | | |
| cysteine present) | | | |
| Suc-Ala-Ala-Pro- | 19.7 +− 0.6 | nda | Nda |
| Phe-AMC (SEQ ID | | | |
| NO: 7) | | | |
| H-Leu-AMC | 61.9 +− 2.7 | 28.4 +− 0.9 | 7.6 +− 3.5 |
| H-Arg-AMC- | nda | nda | nda |
| 2HCl | | | |

nda—no detectable activity.
* (Values indicate mean +− SD residual activity

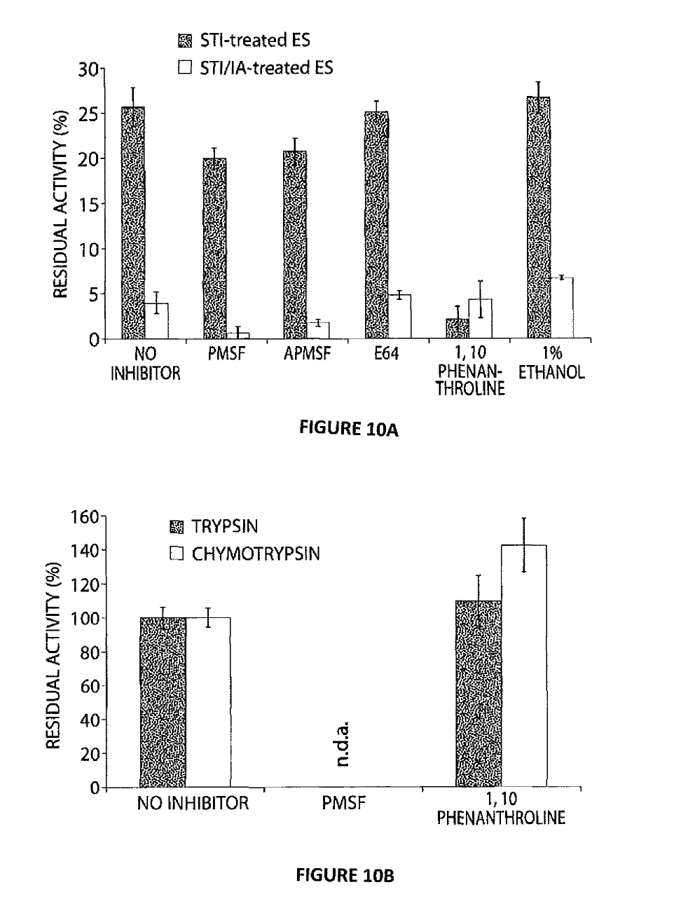

Greatest hydrolysis was observed with Tosyl-Gly-Pro-Arg-AMC HCl. This indicated a predominance of serine proteinase-like activity. The addition of the activator cysteine to the cysteine proteinase and serine proteinase substrate Z-Phe-Arg-AMC HCl did not elicit any increase in substrate hydrolysis. This suggested that the batch of ES tested did not show cysteine proteinase activity, but acted upon the substrate using serine proteinase-like enzyme(s). Its high activity against the serine proteinase substrate Tosyl-Gly-Pro-Arg-AMC HCl indicated existence of such activity within larval ES. Larval ES that had been exposed to soybean trypsin inhibitor or soybean trypsin inhibitor and iminodiacetic acid displayed negligible proteolytic activity against all of the substrates tested, with the exception of the metalloproteinase substrate H-Leu-AMC (Table 2). soybean trypsin inhibitor treated ES displayed 28.4+/−0.9 percent of the activity of untreated ES against H-Leu-AMC, although additional exposure to iminodiacetic acid, a known chelator, further reduced the activity of ES to 7.6+/−3.5 percent of the untreated ES. The activity of ES against the H-Leu-AMC substrate was characterized further using class-specific enzyme inhibitors. As shown in FIG. 10A, the presence of the metalloproteinase inhibitor 1,10 phenanthroline reduced the residual activity of soybean trypsin inhibitor-treated ES from 25.7+/−2.13 percent, when compared with untreated ES, to 2.10+/=1.43 percent. The reduction was significant (p<0.01) using one-way ANOVA and Dunnett's multiple comparison test. This result confirmed the presence of a metalloproteinase within the treated ES. Without wishing to be tied to theory, it is likely that this metalloproteinase is an aminopeptidase. The serine proteinase inhibitors PMSF and APMSF also reduced activity to 19.90+/−1.17 percent and 20.71+/−1.51 percent, respectively. These reductions were significantly different from the values obtained for soybean trypsin inhibitor treated ES in the absence of inhibitor (p<0.01). This indicated the minor presence of a serine proteinase, possibly a trypsin-like enzyme, which displayed some activity against the substrate. In testing the residual activity of soybean trypsin inhibitor/iminodiacetic acid-treated ES, one-way ANOVA and Dunnett's multiple comparison test revealed that the inclusion of 1,10 phenanthroline had no significant effect (p>0.05). This indicated that exposing soybean trypsin inhibitor treated ES to iminodiacetic acid had removed any remaining metalloproteinase activity. PMSF but not APMSF significantly reduced the remaining activity of soybean trypsin inhibitor/iminodiacetic acid-treated ES (p<0.01), indicating the trace presence of a serine proteinase. The inhibitor L-transepoxysuccinyl-leucylamide-(4-guanidino)-butane had no effect upon soybean trypsin inhibitor- or soybean trypsin inhibitor/iminodiacetic acid-treated ES, indicating the lack of cysteine proteinase activity against the substrate. The cross-reactivity of H-Leu-AMC with serine proteinases was determined using commercially available stocks of trypsin and chymotrypsin (FIG. 10B). Trypsin hydrolyzed the substrate, but its specific activity of 0.77+/−0.18 mM/min/mg was just 1.2 percent of the activity of untreated ES (which as shown in Table 2, displayed a specific activity of 61.9-2.7 mM/min/mg), 4.4 percent of the activity of STI-treated ES, and 16.4 percent of soybean trypsin inhibitor/iminodiacetic acid-treated ES. Chymotrypsin displayed a similarly low level of activity (1.04-0.17 mM/min/mg). PMSF blocked all trypsin and chymotrypsin activity against the substrate, whereas 1,10 phenanthroline had no effect, indicating its specificity for metalloproteinases.

### Example 7

### Protease Treatment Reduced the Effect of ES on Cell Migration at Twenty-Four Hours

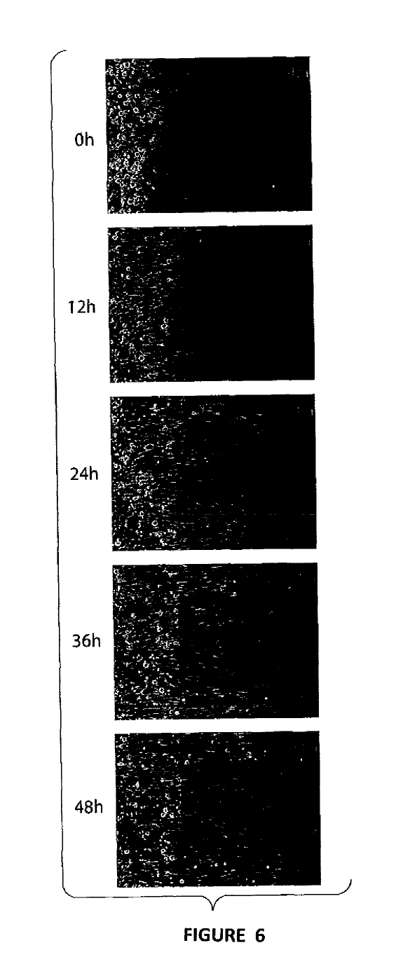

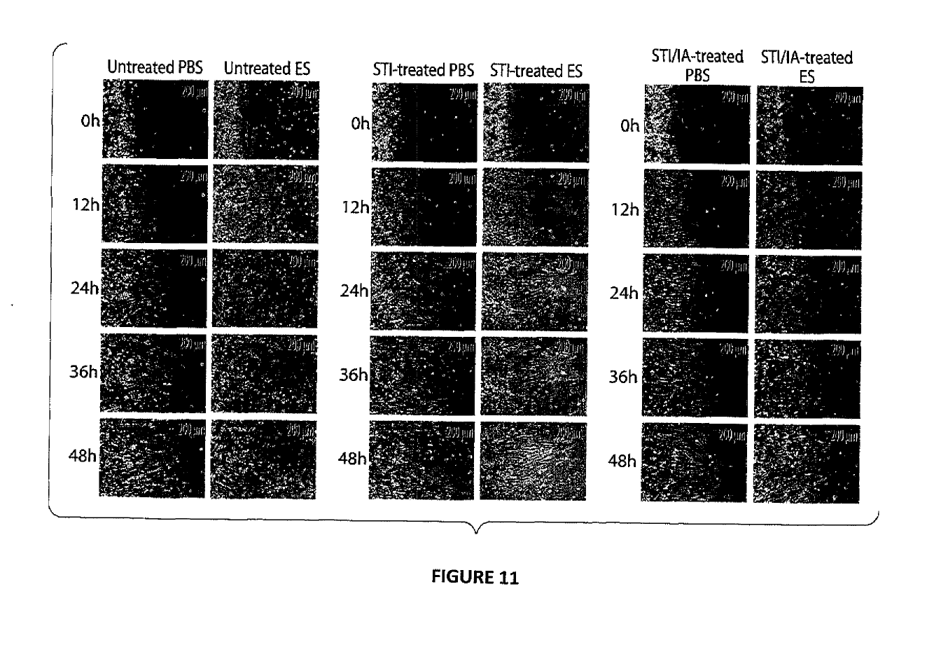

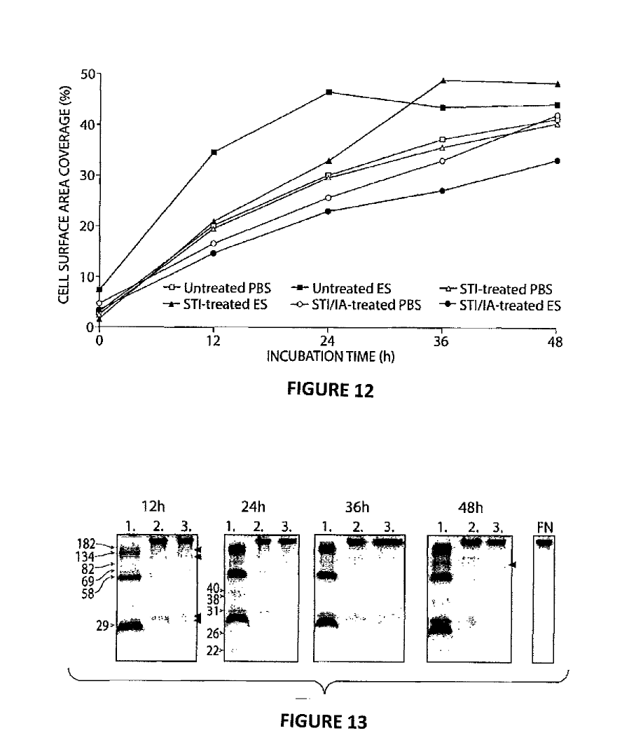

The effect of protease inhibitors on ES-induced cell migration is shown in FIG. 11. A time-lapse movies also showed that among the PBS controls, the rate of HDNF migration across the field of view was similar. The presence of untreated ES promoted cell migration, causing the cells to reach the far right-hand side of the field of view approximately 24 hours earlier than cells within the control. soybean trypsin inhibitor-treated ES exerted little effect upon migration over the first 24 hours of incubation. Throughout this period, the progress of cell movement across the exposed surface was comparable with the progress observed within the control, although after 24 hours, migration appeared to accelerate. Soybean trypsin inhibitor/iminodiacetic acid-treated ES did not enhance HDNF migration across the surface. The cells exhibited similar if not slightly slower progress across the exposed surface than the control. These observations are reflected in the quantitative image analysis data. Here, images displaying the progress of cell migration following 0, 12, 24, 36, or 48 hours of incubation were analyzed, as described in FIG. 6, to assess the percentage area of the surface covered by cells. As shown in FIG. 12, the surface area coverage achieved by cells exposed to 0.1 μg/ml untreated ES attained a plateau of approximately 45 percent by 24 hours of incubation. This result reflects the observation that, by this time, the cells had migrated to the far right-hand side of the field of view. After this time point, cell migration continued off-screen. Up until twenty-four hours of incubation, STI- and STI/IA-treated ES exerted little effect upon migration when compared with the controls, but after this time point, cells that were exposed to STI-treated ES exhibited an acceleration in migration, attaining a plateau of approximately 49 percent cell surface area coverage by thirty-six hours of incubation. By this time, as shown in FIG. 11, the cells had migrated to the far right-hand side of the field of view. Cell surface area coverage in the presence of STI/IA-treated ES remained similar to or lower than the coverage attained under control conditions throughout the incubation period examined.

### Example 8

### ES-Dependent Degradation of Fibronectin

To provide an indication as to whether modification of the fibronectin-coated surface may have played a role in influencing fibroblast migration, a 100-μg/ml solution of bovine fibronectin was exposed to the specified preparation of larval ES (0.1 μg/ml). The solution was resolved through a 12 percent polyacrylamide gel under reducing conditions and the resulting bands visualized by staining. As shown in FIG. 13, after twelve hours of incubation with untreated ES, none of the fibronectin molecule remained intact. It had been extensively degraded into a number of distinct bands ranging from 182 kDa to 29 kDa in size. After 24 hours of incubation, additional bands were present ranging from 40 kDa to 22 kDa in size. After 36 and 48 hours of incubation, the same bands were observed apart from those that represented the two smallest molecular weights (26 kDa and 22 kDa). It is possible that these fragments had been degraded further and run off the gel completely. Limited degradation of fibronectin had occurred in the presence of soybean trypsin inhibitor-treated ES and soybean trypsin inhibitor/iminodiacetic acid-treated ES. Most of the fibronectin molecule had remained intact, although faint bands of 171 kDa, 105 kDa, 32 kDa, and 31 kDa were observed after 12 and 24 hours of incubation. Following thirty-six hours of incubation, the 171-kDa band was no longer visible, and by forty-eight hours of incubation, a band of 79 kDa molecular weight was observed in addition to the bands representing 105 kDa, 32 kDa, and 31 kDa. Samples containing buffer alone or buffer and untreated larval ES (0.1 μg/ml) were also resolved through the gel. After staining, no bands were observed (data not shown).

Thus, it has been shown that ES promotes HDNF migration across a fibronectin-coated surface. Serine proteinase activities within ES have been shown to be responsible and induce migration at least partly through modifying the fibronectin present, although metalloproteinases within ES may also contribute. Thus, a mechanism by which larval ES aids new tissue formation may be via the promotion of fibroblast motility, providing for a wider distribution of viable fibroblasts within the chronic wound due, at least in part, to the action of serine proteinases.

In the clinical setting, maggots are applied to chronic, open ulcers, which most often occur in the elderly. Methods describing such therapy are known in the art and are described, for example, by Bonn et al., Lancet 2000; 356:1174; Church et al., Primary Intent 1999; 7:63-8; Mumcuoglu Am J Clin Dermatol 2001; 2:219-27; and Sherman Annu Rev Entomol 2000; 45:55-81. Fibroblasts taken from chronic open wounds show reduced motility, (Raffetto et al., J Vasc Surg 2001; 33:1233-41) anomalies in matrix metalloproteinases and their specific inhibitor production (Cook et al., J Invest Dermatol 2000; 115:225-33) and characteristics of senescence (Mendez et al., J Vasc Surg 1998; 28:876-83). Senescent fibroblasts, which become prevalent with age, display altered fibronectin expression, defective tissue remodeling, cessation of proliferation, and a subdued response to chemotactic agents (Stanley et al., J Vasc Surg 2001; 33:1206-11; Dimri et al., Proc Natl Acad Sci USA 1995; 92:9363-7; Albini et al., Collagen Rel Res 1988; 1:23-37; Raffetto et al., J Vasc Surg 2001; 34:173-7). The enhancement of fibroblast motility by ES and its modification of ECM proteins, such as fibronectin, may therefore prove beneficial in the chronic wound, modulating the chronic wound fibroblast's behavior. This is particularly so when considering that fibroblast activation appears to be a limiting step of granulation tissue formation (McClain Am J Pathol 1996; 149:1257-70).

### Example 9

### Purification of Trypsin Activity from L. Sericata ES Products

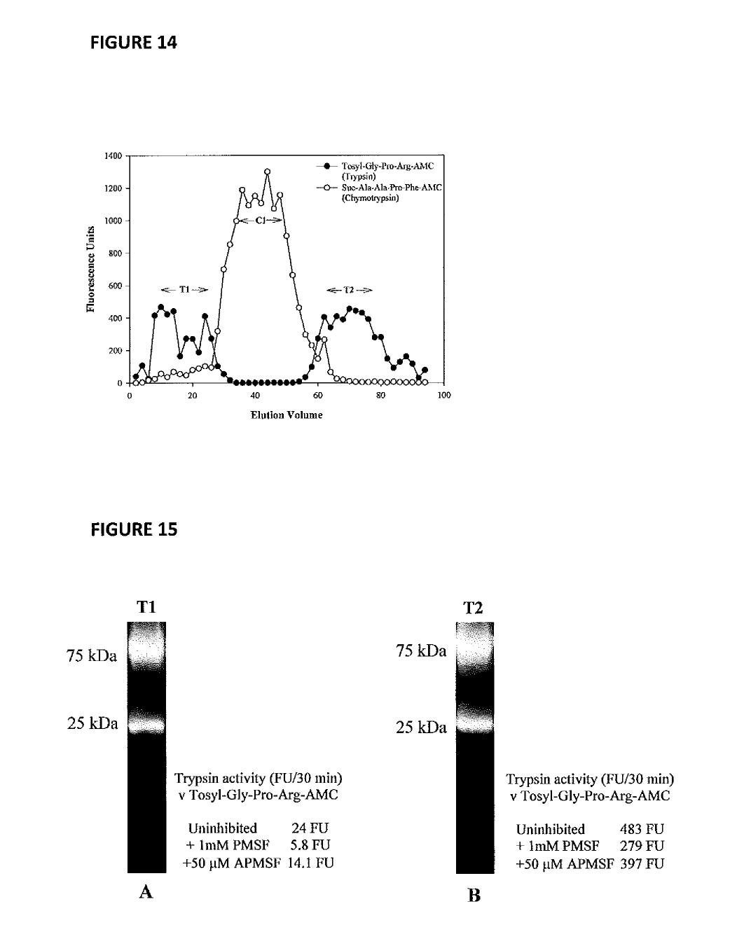

Two fractions containing trypsin activity were purified from L. sericata ES products by a combination of gel filtration using Sephacryl S-300 gel filtration followed by affinity chromatography on p-aminobenzamidine agarose. Following S-300 gel filtration (FIG. 14), 3 peaks of proteolytic activity are observed. A single peak of chymotryptic activity (monitored using the fluorescent substrate Suc-Ala-Ala-Pro-Phe-AMC (SEQ ID NO:7) and currently termed C1) and 2 peaks of tryptic activity (monitored using the fluorescent substrate Tosyl-Gly-Pro-Arg-AMC and termed T1 and T2).

Following S-300 chromatography the T1 and T2 peaks were individually applied to a p-aminobenzamidine agarose column equilibrated with 0.1 M Tris, 0.5 M NaCl, pH8. The column was washed and bound protein eluted with 0.1 M sodium acetate, 0.5 M NaCl, pH 4.0. Fractions eluting from the column were immediately adjusted to pH7 with 1 M Tris HCl pH 9 and assessed for proteolytic activity and inhibitor sensitivity using the substrate Tosyl-Gly-Pro-Arg-AMC and gelatin substrate SDS-PAGE (FIG. 15). The eluted proteins from both columns demonstrate activity typical of trypsin like enzymes cleaving the substrate Tosyl-Gly-Pro-Arg-AMC and being inhibited by both 1 mM PMSF and 50 μM APMSF (FIGS. 15A and B). Trypsin activity is associated with bands of approximately 25 and 75 kDa on gelatin substrate SDS-PAGE.

This shows that there at least two trypsins are present in L. sericata ES.

### Example 10

### Cloning and Expression of L. sericata Trypsin

A cDNA encoding for L. sericata trypsinogen was isolated and inserted into the insect expression vector pBAC-3. This cDNA was obtained by screening an L. sericata cDNA library with primers designed using a trypsin sequence from L. cuprina. The protein sequence of the trypsinogen was determined as SEQ ID NO:1 as shown above.

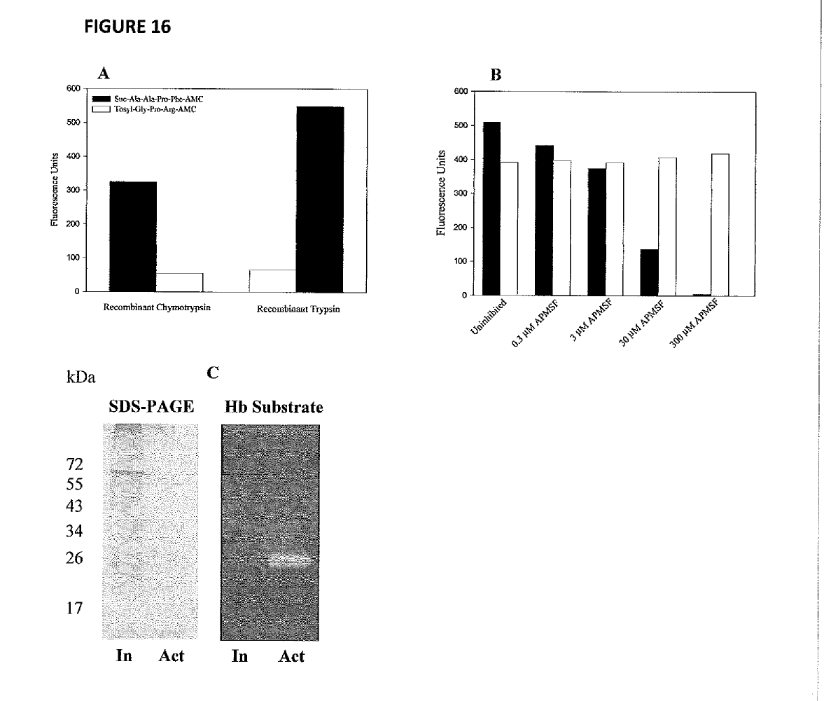

The recombinant protein was expressed by Oxford Expression Technologies. A small scale trial batch of the trypsinogen was activated by incubation with trypsin-agarose for 1 h at 37° C. The trypsin-agarose was removed by centrifugation at 250 g for 5 minutes and proteolytic activity was assessed by monitoring the release of 7-amino-4-methyl coumarin (AMC) from the trypsin substrate Tosyl-Gly-Pro-Arg-AMC and the chymotrypsin substrate Suc-Ala-Ala-Pro-Phe-AMC (SEQ ID NO:7) (FIG. 16A).

The enzymatic activity of the recombinant trypsin was compared with a recombinant chymotrypsin from L. sericata. The recombinant trypsin demonstrated activity against the trypsin substrate Tosyl-Gly-Pro-Arg-AMC in preference to the chymotrypsin substrate Suc-Ala-Ala-Pro-Phe-AMC (SEQ ID NO:7). The recombinant trypsin was also inhibited in a dose dependant manner by the trypsin inhibitor APMSF while the recombinant chymotrypsin remained resistant to all the concentrations tested (FIG. 16B).

The purity and enzymatic activity of the inactive and activated recombinant trypsin was also assessed by SDS-PAGE and haemoglobin substrate SDS-PAGE. The activated trypsin resolved as a protein of approximately 26 kDa corresponding to the predicted molecular mass of 25.9 kDa. Protease activity the same molecular mass was observed on substrate SDS-PAGE (FIG. 16C).

### Example 11

### Trypsin as a Debridement Agent

Degradation of Slough/Eschar from Venous Leg Ulcers by Native Trypsin

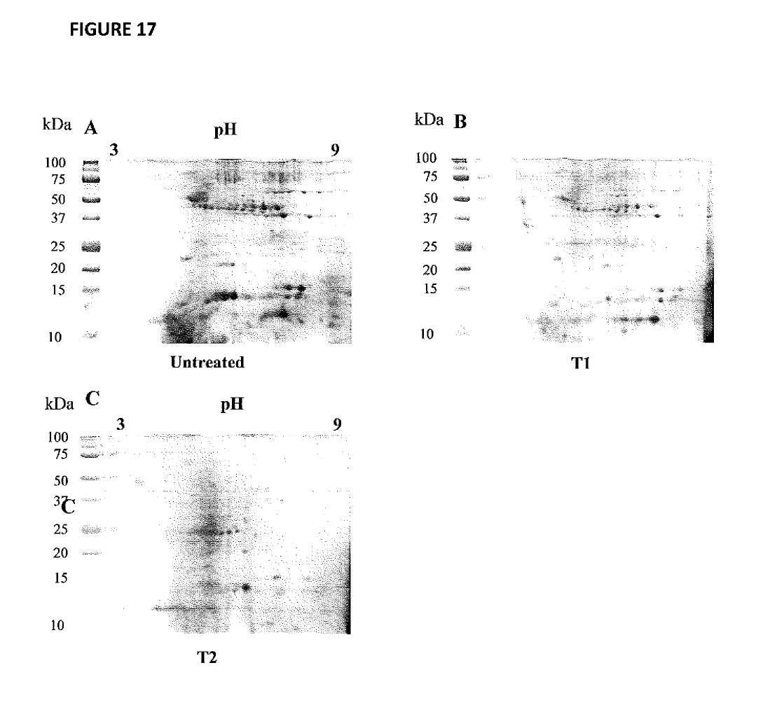

100 μg of untreated wound eschar was incubated with approximately 1 μg of the T1 and T2 trypsin preparations obtained in Example 9 following affinity chromatography on p-aminobenzamidine agarose and incubated overnight at 37° C. 1^{st} dimension electrophoresis was carried out using Biorad pH 3-10 IPG strips. Second dimension electrophoresis was carried out on 10% Tricine gels under reducing conditions. Gels were stained with Coomassie Brilliant Blue R250 (FIG. 6). Compared to an untreated control (FIG. 17A) many of the proteins present in wound eschar were degraded by both trypsin preparations (FIGS. 17B and C) although the T1 preparation was less effective (FIG. 17B) reflecting the weaker proteolytic activity eluting from the p-aminobenzamidine column.

### Example 12

### Degradation of Slough/Eschar by Recombinant L. sericata Trypsin

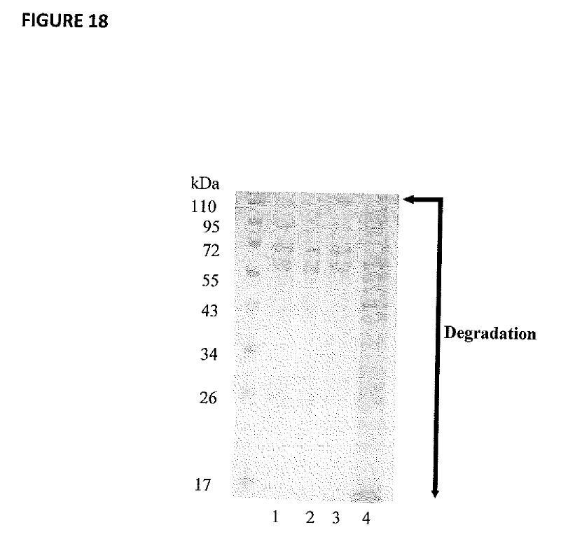

25 μg of eschar was incubated with 10 μl of inactive or activated trypsin obtained as described in Example 10 overnight at 37° C. 10 μl of the activation buffer alone was also included to exclude the possibility that slough/eschar degradation could be due to contaminating trypsin from the activation material. Degradation products were analysed by 12% SDS-PAGE under reducing conditions followed by staining with Coomassie Brilliant Blue R250. Degradation of slough/eschar was only observed in the presence of activated trypsin (FIG. 18, lane 4).

Thus it appears that the recombinant enzyme exhibits the characteristics of the native trypsin cleaving the tryptic substrate Tosyl-Gly-Pro-Arg-AMC SEQ and is inhibited by the inhibitor APMSF. In addition, trypsin obtainable from the trypsinogen of SEQ ID NO:1 degrades wound slough/eschar and so will be useful in therapy.

The results described herein were obtained using the following methods and materials.

### ES Collection

L. sericata secretions or excretions ES were collected from sterile, freshly hatched L. sericata larvae (LarvE, Surgical Materials Testing Laboratory, Cardiff, UK), as previously described by Horobin et al., Br J Dermatol 2003; 148:923-33, which is hereby incorporated by reference in its entirety. Briefly, 400 larvae were washed in 1 mL of sterile phosphate-buffered saline solution (PBS; pH 7.3) for thirty minutes at room temperature to recover ES products. The protein concentration of ES was determined using the Bio-Rad (Hercules, Calif.) protein colorimetric assay, based upon the Bradford method (Bradford, Anal Biochem 1976; 72:248-54). The proteolytic activity of ES was verified using a fluorescein isothiocyanate-casein digest assay (Horobin et al., Br J Dermatol 2003; 148:923-33) and the result was expressed as relative fluorescence per mg of ES protein. Two separate batches of larvae were obtained, and the secretions from each were referred to as Batch A (9.87+/−106 relative fluorescence per mg ES protein) or Batch B (5.07+/−106 relative fluorescence per mg ES protein). Modification of the Proteolytic Activity of L. sericata Larval ES

In some experiments, the larval ES (Batch B) that had been collected was modified before its analysis. For this purpose, a sample of ES was diluted to the equivalent protein concentration of 50 μg/ml using PBS. One aliquot was taken and exposed three successive times to an excess of soybean trypsin inhibitor (STI) immobilized on cross-linked 4% beaded agarose (Sigma-Aldrich, Poole, Dorset, UK). For each exposure, the ES was incubated for approximately thirty minutes at room temperature and gentle agitation applied periodically. Another aliquot was left untreated but kept at room temperature for a similar length of time. Following the final exposure, the ES-STI-agarose suspension was spun, using an MSE Micro Centaur (Sanyo, Loughborough, UK) centrifuge, at 6000 rpm for approximately 30 seconds to pellet the agarose and attached inhibitor. The ES supernatant was recovered and divided into two aliquots. One aliquot was left as it was (STI-treated ES), and the other was exposed two successive times to an excess of iminodiacetic acid (IA) insolubilized on Sepharose 6B Fast Flow (Sigma). Again, each time, the ES was incubated for approximately 30 minutes at room temperature. As before, the insolubilized inhibitor was pelleted by centrifugation and the ES supernatant retained. ES Characterization

The proteolytic activities of the untreated ES, soybean trypsin inhibitor-treated ES, and STI/IA-treated ES were then assessed by monitoring the release of 7-amino-4-methylcoumarin (AMC) from the synthetic peptide substrates Tosyl-Gly-Pro-Arg-AMC.HCl, Suc-Ala-Ala-Pro-Phe-AMC (SEQ ID NO:7), Z-Phe-Arg-AMC.HCl, H-Pro-Phe-Arg AMC.2HCl, Z-Gly-Gly-Arg-AMC.HCl, H-Leu-AMC, and H-Arg-AMC.2HCl (Table 1).

TABLE 1
Enzyme class specificity for
fluorogenic peptide substrates

| Peptide substrate | Enzyme |
|---|---|
| Tosyl-Gly-Pro-Arg-AMC-HCl | Thrombin and plasmin |
| | (serine proteinase) |
| H-Pro-Phe-Arg AMC-2HCl | Kallikreins and elastase |
| | (serine proteinase) |
| Z-Gly-Gly-Arg-AMC-HCl | Urokinase (serine proteinase) |
| Z-Phe-Arg-AMC-HCl | Papsin (cysteine proteinase) |
| | and trypsin (serine proteinase) |
| Suc-Ala-Ala-Pro-Phe-AMC | Chymotrypsin |
| (SEQ ID NO: 7) | (serine proteinase) |
| H-Leu-AMC | Leucine aminopeptidase |
| | (metalloproteinase) |
| H-Arg-AMC-2HCl | Cathepsin (cysteine |
| | proteinase) and aminopeptidase |
| | (metalloproteinase) B |

An aliquot of the treated or untreated ES was diluted to 0.2 μg/ml with PBS and added to the peptide substrate, which had also been diluted with PBS, to give a final concentration of 5 mM substrate and 3.3×10⁻² μg/mL ES. Where specified, before the addition of the H-Leu-AMC substrate, a class-specific proteinase inhibitor was preincubated with the STI- or STI/IA-treated ES for 10 minutes. Substrate hydrolysis was performed in the presence of 1 mM phenylmethylsulfonyl fluoride (PMSF), 100 mM 4-(amidinophenyl)methanesulfonyl fluoride (APMSF), 5 mM L-trans-epoxysuccinyl-leucylamide-(4-guanidino)-butane, or 10 mM 1,10 phenanthroline. PMSF and 1,10 phenanthroline were dissolved in dilute ethanol, giving a final concentration of 1% ethanol during substrate hydrolysis. Hence, the effect of 1% ethanol on substrate hydrolysis was also tested. In addition to ES, the H-Leu-AMC substrate was hydrolyzed by a similar concentration of bovine trypsin (Sigma) or bovine □-chymotrypsin (Sigma) to clarify substrate specificity. Where indicated, a class-specific proteinase inhibitor was also introduced, as described above. Fluorescence was detected at 365 nm excitation/465 nm emission wavelengths (Dynex MFX microtiter plate fluorometer, Chantilly, Va.). Fluorescence emitted from hydrolysis of the cysteine proteinase substrate Z-Phe-Arg-AMC.HCl was monitored in the presence and absence of 5 mM cysteine, a cysteine proteinase activator. Samples containing PBS only were left untreated or treated with STI and STI/IA, following the protocol described for the treatment of larval ES. The PBS samples were then tested against the synthetic peptide substrates, as described above for ES, to provide baseline measurements of autohydrolytic activity. They were also incorporated into the cell migration assays to act as controls.

### Fibroblast Cell Culture

Human, dermal, neonatal fibroblast (HDNF) cells (TCS Cellworks, Buckingham, UK) were grown in T75 flasks (Nunc, Life Technologies Ltd, Paisley, UK), containing standard cell culture medium comprising Dulbecco's Modified Eagle's Medium (Gibco Invitrogen Ltd, Paisley, UK), 10 percent fetal calf serum (FCS; Sigma), antibiotic/antimycotic solution (Sigma; 100 units/mL penicillin, 100 μg/ml streptomycin, and 0.25 μg/ml amphotericin B), and 2 mM L-glutamine (Sigma). For experiments, HDNF cells were trypsinized and transferred to FCS-free cell culture medium. Cells were incubated at 37° C. in a 5% (v/v) CO2 humidified atmosphere. HDNF cells of passage number 6 or 9 were included within preliminary work that first explored the effects of unmodified larval ES upon cell migration. Additional experiments investigating the effects of larval ES that had been modified to alter its proteolytic activity included cells that were all derived from the same initial source and were all of passage number 7.

### Two-Dimensional In Vitro Wound Assay

A 35-mm tissue culture dish (Nunc) containing 2 mL of bovine fibronectin (Sigma), diluted with PBS to 100 mg/mL, was incubated overnight at 37 [deg.] C. Remaining fibronectin solution was aspirated, and a sterile, glass cloning cylinder of 4.7 mm internal diameter (Sigma) was placed upright in the middle of the dish. HDNF cells (1×10⁶) were seeded around the outside of the cylinder together with ES or, for the control, an equivalent volume of PBS. In the preliminary work, an appropriate volume of ES from Batch A was added to the cells to yield a final concentration of 0.1 μg/ml ES. Additional investigations incorporated an appropriate volume of 50 μg/ml ES (from Batch B) that had previously been exposed to STI and STI plus IA or left untreated, such that a final concentration of 0.1 μg/ml ES was obtained with the cells. ES was also added to the inside of the cloning cylinder, maintaining exposure of the whole fibronectin-coated surface to 0.1 μg/ml ES. Where ES was absent, an equivalent volume of PBS, previously exposed to the aforementioned inhibitors or left untreated, was added in its place. After the cells had been maintained at 37° C. for 4 hours, the cloning cylinder was removed, taking care not to disturb the confluent cell layer that had formed around it. The dish was then immediately placed under a Leica (Cambridge, UK) DM IRBE inverted microscope and positioned so that the inner boundary of the cell layer was viewed in a vertical orientation and approximately one-third of the field of view was taken up by cells. A temperature of 37° C. was maintained using a heated Perspex incubation chamber surrounding the stage, and sterile filtered 5% (v/v) CO2 was perfused over the dish. Digital images were then taken of the same field of view at specified intervals over a 48-hour period. The point at which the first digital image was recorded was taken as 0 hours incubation. For the preliminary work, images were taken every 3 minutes using a JVC TK C1380 camera in conjunction with Lida (Leica) software. For additional studies, images were taken every 6 minutes using a SPOT Insight camera (Diagnostic Instruments Inc., Sterling Heights, Mich.) in conjunction with Advanced SPOT software. The images were then sorted in ascending chronological order and compiled into a Microsoft AVI movie format using Adobe Premiere 5.1 (San Jose, Calif.). Movies may be downloaded and viewed from the Wound Repair and Regeneration Web site vw.cphs.wayne.edu/wrr/DOWNLOADS.HTM. Cell

### Cell Proliferation Assay

An ATP assay was used to confirm that the concentration of larval ES present within the two-dimensional in vitro wound assays did not influence HDNF cell proliferation. The ATPLite-M assay system (PerkinElmer, Boston, Mass.) monitors adenosine triphosphate (ATP) levels by using the reaction of ATP present within a sample with added luciferase and D-luciferin. Preparatory work confirmed that the intensity of luminescence detected from the ATP assay displayed a linear relationship with fibroblast cell density (1500-150,000 cells/mL; data not shown). It was also confirmed that the presence of ES did not influence the ATP levels detected from samples containing known concentrations of the ATP standard. For experiments, 100 ml of fetal calf serum (FCS)-free cell culture medium, containing 45,000 cells/mL and the specified concentration of larval ES, was added to each well of an opaque, white 96-well plate (Nunc) that had previously been coated with fibronectin. For this purpose, 100 ml of bovine fibronectin (Sigma), diluted to 100 mg/mL using PBS, was added to each well and incubated overnight at 37 [deg.] C. Wells were aspirated of remaining fibronectin solution and left to air dry before the addition of cells. The cells were incubated at 37 [deg.] C. for the time stated and the ATP measured according to the manufacturer's protocol. Results were converted to ATP concentration by comparison with data taken from an ATP dilution.

### Degradation of Fibronectin

The proteolytic degradation of fibronectin by ES was assessed using sodium dodecyl sulfate-polyacrylamide gel electrophoresis (Laemmli Nature 1970; 227:680-5). Here, bovine fibronectin (Sigma), diluted with PBS to 100 μg/ml, was incubated at 37 [deg.] C. for the time stated in the absence or presence of 0.1 μg/ml untreated ES, STI-treated ES, or STI/IA-treated ES. At each specified incubation time point, an aliquot, containing 40 μg fibronectin, was taken and precipitated in 80% (v/v) ice-cold acetone. The precipitate was redissolved in distilled water containing 1% (v/v) Strataclean (Stratagene, La Jolla, Calif.), which selectively binds protein. After 5 minutes at room temperature, this mixture was pelleted by centrifugation, suspended in reducing sample buffer, and heated to 100° C. for 5 minutes. Samples were then resolved through a 12 percent polyacrylamide gel. Protein bands were detected by staining with 0.25% Coomassie Brilliant Blue R250 in 25% methanol, 10% acetic acid, followed by destaining in the same solvent without the Coomassie stain.

### Analysis of Results

In addition to qualitative analysis of the time-lapse movies, separate images, taken at the specified incubation time, were analyzed quantitatively for the extent of cell migration into the free space created by the cloning cylinder. This was undertaken using Leica QUIPS software. As shown in FIG. 6, a rectangular area within the image, beginning at the edge of the cell boundary at time 0 hours and ending at the far right-hand side of the image (an initially cell-free area) was defined. In both the preliminary work and additional studies, the defined area was kept at the same size and position for all images analyzed. The outline of each cell lying within the defined rectangular area was then traced, and the total area within the rectangle covered by cells was calculated and expressed as a percentage of the whole rectangular area. Results were analyzed for statistically significant differences using GraphPad Prism (San Diego, Calif.) software. Statistical significance was taken as p-0.05. A two-way analysis of variance (ANOVA) test was applied to the ATP-based cell proliferation assay results. Values representing the activity of STI- or STI/IA-treated ES against the H-Leu-AMC substrate, in the presence or absence of the specified proteinase inhibitor, were analyzed using one-way ANOVA tests incorporating Dunnett's multiple comparisons.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An isolated polypeptide selected from the group consisting of
(a) a trypsinogen of SEQ ID NO: 5 MKTFIALSLFVAIASAGILPAVEQRLPLVPVMPLEELEGRITNGELAKPGQFPYQA GLSLVFGNKGAWCGGTLISNRWVLTAAHCTDGADGVTVYLGATDIKNDNEKGQ QRIYSSKANIVVHANWDASTLSNDISLIKLPVAVEFNELIQPATLPKMDGKYSTYE GDMVWASGWGRDSDAATSVSQFLRYIEVPVLKQSTCKTYYLGSVTDKMICIKSV DKKSTCNGDSGGPLVYKDGGVNYVIGATSFGIALGCEKGWPGVFTRVTSYLDWI EEVSGVVNK (SEQ ID NO:5);
(b) or a functional homologue thereof having at least 96% identity to SEQ ID NO:5, or
(c) a trypsin derivable from any of (a) or (b), or
(d) a fragment of any of these having trypsin activity.

2. The isolated polypeptide of claim 1 comprising a trypsin derivable from the trypsinogen of SEQ ID NO:5 which is of SEQ ID NO:6.

3. A nucleic acid that encodes a polypeptide according to claim 1.

4. The nucleic acid of claim 3 that encodes a trypsinogen of SEQ ID NO:5 or a functional homologue having at least 96% identity to SEQ ID NO:5.

5. An expression vector comprising a nucleic acid according to claim 4.

6. A method for preparing a polypeptide according to claim 1, the method comprising transforming a cell with an expression vector according to claim 5, causing the cell to express a polypeptide encoded by said vector and recovering trypsinogen produced thereby.

7. The method of claim 6, which further comprises the step of activating trypsinogen recovered to form a trypsin.

8. The method of claim 7, wherein the trypsinogen is activated by incubation with trypsin-agarose.

9. A pharmaceutical composition for promoting wound healing, the composition comprising an effective amount of a polypeptide or peptide fragment of claim 1 and a pharmaceutically acceptable excipient.

10. The composition of claim 8, wherein the composition comprises a sterile support that provides a dressing to protect the wound.

12. The composition of claim 8, wherein the polypeptide is a trypsin.

13. A method for promoting wound healing in a subject in need thereof, the method comprising contacting the wound with an effective amount of a polypeptide of claim 1.

14. The method of claim 13, wherein the method comprises the debridement of the wound and tissue regeneration by the promotion of cell migration conducive to healing.

15. A packaged pharmaceutical comprising:
a) a polypeptide of claim 1; and
b) instructions for using said polypeptide to promote or enhance wound healing in a subject.

16. A kit for promoting or enhancing wound healing in a subject comprising:
a) a polypeptide of claim 1; and
b) instructions for using said polypeptide to promote or enhance wound healing in a subject.

17. A polypeptide obtainable from excretions/secretions of Lucilia sericata that has a molecular weight of 75 kDa and is effective as a trypsin.
